(19) United States
(12) Patent Application Publication
(10) Pub. No.: US 2003/0035829 A1
(43) Pub. Date: Feb. 20, 2003

(54) LIPOSOMAL COMPOSITIONS FOR THE DELIVERY OF NUCLEIC ACID CATALYSTS

(75) Inventors: SEAN C. SEMPLE, VANCOUVER (CA); SANDRA K. KLIMUK, NORTH VANCOUVER (CA); PETER SCHERRER, VANCOUVER (CA); MICHAEL J. HOPE, VANCOUVER (CA); YUAN-PENG ZHANG, VANCOUVER (CA); MARK REYNOLDS, LAFAYETTE, CO (US); JOHN MIN, BOULDER, CO (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: TOWNSEND AND TOWNSEND AND CREW

(21) Appl. No.: 09/122,588

(22) Filed: Jul. 23, 1998

Related U.S. Application Data
(60) Provisional application No. 60/053,813, filed on Jul. 24, 1997.

(51) Int. Cl. A61K 48/00; A61K 9/127; C12N 15/88

(52) U.S. Cl. 424/450; 435/458; 514/44

(57) ABSTRACT

The present invention relates to compositions and methods for delivering nucleic acid catalysts e.g., vascular endothelial growth factor receptor (VEGF-R-1) ribozyme, into a biological system.

### FIELD OF THE INVENTION

The present invention relates to compositions and methods for delivering nucleic acid catalysts, e.g., a vascular endothelial growth factor receptor (VEGF-R-1) ribozyme, into a biological system.

### BACKGROUND OF THE INVENTION

Catalytic nucleic acid molecules (ribozymes) are nucleic acid molecules capable of catalyzing one or more of a variety of reactions, including the ability to repeatedly cleave other separate nuclear, acid molecules in a nucleotide base sequence-specific manner. Such enzymatic nucleic acid molecules can be used, for example, to target cleavage of virtually any RNA transcript (Zaug, et al., Nature, 324:429, 1986; Cech, JAMA, 260:3030, 1988; and Jefferies, et al., Nucleic Acids Research, 17:1371, 1989). Catalytic nucleic acid molecules mean any nucleotide base-comprising molecule having the ability to repeatedly act on one or more types of molecules, including but not limited to enzymatic nucleic acid molecules. By way of example but not limitation, such molecules include those that are able to repeatedly cleave nucleic acid molecules, peptides, or other polymers, and those that are able to cause the polymerization of such nucleic acids and other polymers. Specifically, such molecules include ribozymes, DNAzymes, external guide sequences and the like. It is expected that such molecules will also include modified nucleotides compared to standard nucleotides found in DNA and RNA.

Because of their sequence-specificity, trans-cleaving enzymatic nucleic acid molecules show promise as therapeutic agents for human disease (Usman & McSwiggen, 1995, Ann. Rep. Med. Chem., 30:285-294; Christoffersen and Marr, 1995, J. Med. Chem., 38:2023-2037). Enzymatic nucleic, acid molecules can be designed to cleave specific RNA targets within the background of cellular RNA. Such a cleavage event renders the RNA non-functional and abrogates protein expression from that RNA. In this manner, synthesis of a protein associated with a disease state can be selectively inhibited. In addition, enzymatic nucleic acid molecules can be used to validate a therapeutic gene target and/or to determine the function of a gene in a biological system (Christoffersen, 1997, Nature Biotech., 15:483).

There are at least seven basic varieties of enzymatic RNA molecules derived from naturally occurring self-cleaving RNAs. Each can catalyze the hydrolysis of RNA phosphodiester bonds in trans (and thus can cleave other RNA molecules) under physiological conditions. In general, enzymatic nucleic acids act by first binding to a substrate/target RNA. Such binding occurs through the substrate/target binding portion of an enzymatic nucleic acid molecule which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic and selective cleavage of such a target RNA will destroy its ability to direct synthesis of an encoded protein. After an enzymatic nucleic acid has bound and cleaved its RNA target, it is released from that RNA to search for another target and thus can repeatedly bind and cleave new targets.

The enzymatic nature of a ribozyme is advantageous over other technologies, since the effective concentration of ribozyme sufficient to effect a therapeutic treatment is generally lower than that of an antisense oligonucleotide. This advantage reflects the ability of the ribozyme to act enzymatically. Thus, a single ribozyme (enzymatic nucleic acid) molecule is able to cleave many molecules of target RNA. In addition, the ribozyme is a highly specific inhibitor, with the specificity of inhibition depending not only on the base-pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in basepairing. Thus, it is thought that the specificity of action of a ribozyme is greater than that of antisense oligonucleotide binding the same RNA site.

Trafficking of large, charged molecules into living cells is highly restricted by the complex membrane systems of the cell. Specific transporters allow the selective entry of nutrients or regulatory molecules, while excluding most exogenous molecules such as catalytic nucleic acids. The two major strategies for improving the transport of catalytic nucleic acids into cells are the use of vectors or lipid compositions. Vectors, such as viral vectors, can be used to transfer genes efficiently into some cell types, but they cannot be used to introduce chemically synthesized molecules into cells. An alternative toxicity approach is to use delivery formulations incorporating lipid such as cationic lipids, which interact with nucleic acids through one end and lipids or membrane systems through another (for a review see, Felgner, 1990, Advanced Drug Delivery Reviews, 5:162-187; Felgner, 1991, J. Liposome Res., 3:3-16). Synthetic nucleic acids as well as plasmids may be delivered using known cytofectins, although their utility is often limited by cell-type specificity, requirement for low serum during transfection, and toxicity.

Since the first description of liposomes in 1965, by Bangham (J. Mol. Biol., 13:238-252), there has been a sustained interest and effort in the area of developing lipid-based carrier systems for the delivery of pharmaceutically active compounds. Liposomes are attractive drug carriers since they protect the biological from nuclease degradation while improving their cellular uptake.

One of the most commonly used classes of liposome formulations for delivering polyanions (e.g., DNA) are those that contain cationic lipids. Lipid aggregates can be formed with macromolecules using cationic lipids alone or including other lipids and amphiphiles such as phosphatidylethanolamine. It is well known in the art that both the composition of the lipid formulation as well as its method of preparation have effect on the structure and size of the resultant anionic macromolecule-cationic lipid. These factors can be modulated to optimize delivery of polyanions to specific cell types in vitro and in vivo. The use of cationic lipids for cellular delivery of biopolymers has several advantages. The encapsulation of anionic compounds using cationic lipids is essentially quantitative due to electrostatic interaction. In addition, it is believed that the cationic lipids interact with the negatively charged cell membranes initiating cellular membrane transport (Akhtar, et al., 1992, Trends Cell Bio., 2:139; Xu, et al., 1996, Biochemistry, 35:5616).

The transmembrane movement of negatively charged molecules such as nucleic acids may therefore be markedly improved by co-administration with cationic lipids or other permeability enhancers (Bennett, et al., 1992, Mol. Pharmacol., 41:1023-33; Capaccioli, et al., 1993, BBRC, 197:818-25; Ramila, et al., 1993, J. Biol. Chem., 268:16087-16090. Stewart, et al., 1992, Human Gene Therapy, 3:267-275). Since the introduction of the cationic lipid DOTMA and its liposomal formulation Lipofectin® (Felgner, et al., 1987, PNAS, 84:7413-7417; Eppstein, et al., U.S. Pat. No. 4,897,355), a number of other lipid-based delivery agents have been described primarily for transfecting mammalian cells with plasmids or antisense molecules (Rose, U.S. Pat. No. 5,279,833; Eppand, et al., U.S. Pat. No. 5,283,195; Gebeyehu, et al., U.S. Pat. No. 5,334,761; Nantz, et al., U.S. Pat. No. 5,527,928; Bailey, et al., U.S. Pat. No. 5,552,155; Jesse, U.S. Pat. No. 5,578,475). However, each formulation is of limited utility because it can deliver plasmids into some but not all cell types, usually in the absence of serum (Bailey, et al., 1997, Biochemistry, 36:1628). Concentrations (charge and/or mass ratios) that are suitable for plasmid delivery (˜5,000 to 10,000 bases in size) are generally not effective for oligonucleotides such as synthetic ribozyme molecules (˜10 to 50 bases) (Sullivan, 1993, Meth. Enzy., 5:61-66). Also, recent studies indicate that optimal delivery conditions for antisense oligonucleotides and ribozymes are different, even in the same cell type (Jarvis, et al., 1996, RNA, 2:419; Jarvis, et al., 1996, J. Biol. Chem., 271:29107). However, the number of available delivery vehicles that may be utilized in the screening procedure is highly limited, and there continues to be a need to develop transporters that can enhance nucleic acid entry into many types of cells.

Eppstein, et al., U.S. Pat. No. 5,208,036, disclose a liposome, LIPOFECTIN™ that contains an amphipathic molecule having a positively charged choline head group (water soluble) attached to a diacyl glycerol group (water insoluble). LIPOFECTIN™ has been used to deliver ribozymes to cells (Sioud, et al., 1992, J. Mol. Bio., 223:831; Jarvis, et al., 1996, supra). GIBCO-BRL markets another cationic lipid, LipofectAMINE™, which can help introduce catalytic nucleic acid molecules into certain cells (Jarvis, et al., 1996, supra).

Wagner, et al., 1991, Proc. Nat. Acad. Sci. USA, 88:4255; Cotten, et al., 1990, Proc. Nat. Acad. Sci. USA, 87:4033; Zenke, et al., 1990, Proc. Nat. Acad. Sci. USA, 87:3655; and Wagner, et al., Proc. Nat. Acad. Sci. USA, 87:3410), describe transferrin-polycation conjugates which may enhance uptake of DNA into cells. They also describe the feature of a receptor-mediated endocytosis of transferrin-polycation conjugates to introduce DNA into hematopoietic cells.

Wu, et al., J. Biol. Chem., 266:14338; describe in vivo receptor-mediated gene delivery in which an asialoglycoprotein-polycation conjugate consisting of asialoorosomucoid is coupled to poly-L-lysine. A soluble DNA complex was formed capable of specifically targeting hepatocytes via asialoglycoprotein receptors present on the cells.

Hudson, et al., 1996, Int. J. Pharmaceutics, 136:23; describe the use of thin film poly-(L-lactic acid) (PLA) matrices to deliver ribozymes to cells. The authors reported that the PLA-entrapped ribozymes provided improved biological stability and sustained delivery of ribozymes.

Biospan Corporation, International PCT Publication No. WO 91/18012, describe cell internalizable covalently bonded conjugates having an “intracellularly cleavable linkage” such as a “disulfide cleavable linkage” or an enzyme labile ester linkage.

Choi, et al., 1996, International PCT Publication No. WO 96/10391, describe polyethylene glycol (PEG)-modified lipids and liposomes for the delivery of biological agents including, for example, nucleosides, DNA plasmids and oligonucleotides.

Ansell, et al., 1996, International PCT Publication No. WO 96/10390, describe liposome compositions including a cationic lipid and a neutral lipid to deliver DNA and RNA molecules.

### SUMMARY OF THE INVENTION

The present invention relates to compositions and methods for delivering nucleic acid catalysts, e.g., vascular endothelial growth factor receptor (VEGF-R-1) ribozymes, to a biological system. More particularly, the present invention relates to compositions for delivering nucleic acid catalysts to a cell, the composition comprising a lipid, a polyethyleneglycol-ceramide (PEG-Cer) conjugate and a nucleic acid catalyst (e.g., a VEGF-R-1 ribozyme). In a presently preferred embodiment, the composition comprises a non-cationic lipid, a cationic lipid, a polyethyleneglycol-ceramide (PEG-Cer) conjugate and a nucleic acid catalyst (e.g., a VEGF-R-1 ribozyme). Such compositions have improved circulation characteristics and serum-stability and, thus, can be used to deliver nucleic acid catalysts to cells both in vitro and in vivo, and in the presence or absence of serum.

As a result of their enhanced circulation characteristics, the compositions of the present invention allow for the effective systemic administration of nucleic acid catalysts to a whole animal, thereby providing therapeutically effective means for the treatment of various diseases, such as inflammation, cancer, tumor angiogenesis, infectious diseases, tumor metastasis and others. The compositions of the present invention are particularly useful for modulating angiogenesis, reducing tumor density and decreasing tumor metastasis. As such, the compositions and methods of the present invention can be used to administer, preferably systemically, PEG-Cer formulated nucleic acid catalysts compositions in amounts sufficient to achieve the delivery of the nucleic acid catalysts to the biological system of interest for the treatment of various diseases.

As noted above, in one embodiment, the compositions of the present invention comprise, inter alia, a lipid, a PEG-Cer conjugate and a nucleic acid catalyst. Numerous lipids can be used in the compositions of the present invention. In preferred embodiments, the lipid is a diacylphosphatidylcholine and, in particular, egg yolk phosphatidylcholine (EYPC). In addition, the compositions of the present invention comprise a cationic lipid. Numerous cationic lipids can be used in the compositions of the present invention. In preferred embodiments, the cationic lipid is N,N-dioleyl-N,N-dimethylammonium chloride (DODAC) or 1,2-dioleoyloxy-3-(N,N,N-trimethylamino)propane chloride (DOTAP). In addition, the compositions of the present invention contain a PEG-Cer conjugate having fatty acid groups of various chain lengths. Preferably, the ceramide has a fatty acid group having between 6 and 24 carbon atoms. In particularly preferred embodiments, the PEG-Cer conjugate has fatty acid groups comprising 8, 14, or 20 carbon atoms, designated as PEG-Cer-C8 (or PEG-C8), PEG-Cer-C14 (or PEG-C14); and PEG-Cer-C20 (or PEG-C20), respectively. In a preferred embodiment, the compositions of the present invention comprise, inter alia, a non-cationic lipid (e.g., a diacylphosphatidylcholine), a cationic lipid (e.g., DODAC, DOTAP, etc.), a PEG-Cer conjugate and a nucleic acid catalyst.

In a preferred embodiment, the nucleic acid catalyst used in the compositions of the present invention has an endonuclease activity. Preferably, the nucleic acid catalyst is capable of cleaving a separate nucleic acid molecule and, preferably, the separate nucleic acid molecule is an RNA molecule. More preferably, the target RNA is involved in a mammalian disease. In one embodiment of the invention, the nucleic acid catalyst is targeted to cleave RNA encoded by vascular endothelial growth factor (VEGF) receptor (VEGF-R) genes.

In preferred embodiments, the composition of the present invention contain one or more additional components. One preferred additional component is cholesterol, which can be added to increase the thermal transition temperature of the lipid bilayer, for example, in cases where it is necessary to increase the stability of the liposome in a biological system and/or to reduce the rate of leakage of encapsulated enzymatic nucleic acid. Another preferred additional component is a lipid, such as a pH-sensitive lipid, which may be added to increase the amount of nucleic acid catalyst (e.g., VEGF-R-1 ribozyme) that can be encapsulated in the formulation.

In yet another preferred embodiment, the compositions of the present invention comprises diacylphosphatidylcholine (e.g., egg yolk phosphatidylcholine), a PEG-Cer conjugate, a cationic lipid (e.g., DODAC or DOTAP) and a nucleic acid catalyst. As described herein, the various components of the compositions of the present invention are combined in proportions suitable for the delivery of nucleic acid catalysts to a desired cell or biological system of interest.

In another embodiment, the present invention provides pharmaceutical compositions comprising at least one PEG-Cer formulated nucleic acid catalyst and a pharmaceutically or veterinerially acceptable carrier. Such pharmaceutical compositions can effectively be used for the treatment of human diseases, such as cancer, inflammation, tumor angiogenesis, tumor metastasis, ocular diseases and the like.

In a preferred embodiment, the invention provides PEG-Cer formulated nucleic acid catalyst compositions, wherein the nucleic acid catalyst (e.g., a VEGF-R-1 ribozyme) is capable of decreasing expression of RNA associated with a mammalian disease, for example, a human disease such as cancer or inflammation.

In another embodiment, the invention provides methods of facilitating the transfer of a nucleic acid catalyst into a target cell, the method comprising the step of contacting the target cell with the PEG-Cer formulated nucleic acid catalyst composition under conditions suitable for the transfer of the nucleic acid catalyst into the cell.

In yet another embodiment, the invention provides methods for treating numerous diseases (e.g., cancer or inflammation) in a patient, the methods comprising the step of administering (e.g., systemically or locally) to the patient a PEG-Cer formulated nucleic acid composition under conditions in which expression of the RNA associated with the disease is decreased in the patient and a therapeutic result is attained. As such, the methods of the present invention allow for the local administration (e.g., ocular administration) of a PEG-Cer formulated nucleic acid composition as well as for the systemic administration of a PEG-Cer formulated nucleic acid composition.

Other features, objects and advantages of the invention and its preferred embodiments will become apparent from the detailed description which follows.

### BRIEF DESCRIPTION OF THE DRAWINGS

FIG. 1 is illustrates the secondary structure model for seven different classes of enzymatic nucleic acid molecules. Arrows indicate the site of cleavage. --------- indicate the target sequence. Lines interspersed with dots are meant to indicate tertiary interactions. - is meant to indicate base-paired interaction. Group I Intron: PI-P9.0 represent various stem-loop structures (Cech, et al., 1994, Nature Struc. Bio., 1, 273). RNase P (MIRNA): EGS represents external Code sequence (Forster, et al., 1990, Science, 249, 783; Pace, et al., 1990, J. Biol. Chem., 265, 3587). Group II Intron: 5′SS means 5′ splice size; 3′SS means 3′-splice site; IBS means intron binding site; EBS means exon binding site (Pyle, et al., 1994, Biochemistry, 33, 2716). VS RNA: I-VI are meant to indicate six stem-loop structures; shaded regions are meant to indicate tertiary interaction (Collins, International PCT Publication No. WO 96/19577). HDV Ribozyme: I-IV are meant to indicate four stem-loop strucnires (Been, et al., U.S. Pat. No. 5,625,047). Hammerhead Ribozyme: I-III are meant to indicate three stem-loop structures; stems I-III can be of any and may be symmetrical or asymmetrical (Usman, et al., 1996, Curr. Op. Struct. Bio., 1, 527). Hairpin Ribozyme: Helix 1, 4 and 5 can be of any length; Helix 2 is between 3 and 8 base-pairs long; Y is a pyrimidine; Helix 2 (H2) is provided with a least 4 base pairs (i.e., n is 1, 2, 3 or 4) and helix 5 can be optionally provided of length 2 or more bases (preferably, 3-20 bases, i.e., m is from 1-20 or more). Helix 2 and helix 5 may be covalently linked by one or more bases (i.e., r is≧1 base). Helix 1, 4 or 5 may also be extended by 2 or more base pairs (e.g., 4-20 base pairs) to stabilize the ribozyme structure, and prefably is a protein binding site. In each instance, each N and N′ independently is any normal or modified base and each dash represents a potential base-pairing interaction. These nucleotides may be modified at the sugar, base or phosphate. Complete base-pairing is not required in the helices, but is preferred. Helix 1 and 4 can be of any size (i. e., o and p is each independently from 0 to any number, e.g., 20) as long as some base-pairing is maintained. Essential bases are showm as specific bases in the structure, but those in the art will recognize that one or more may be modified chemically (abasic, base, sugar and/or phosphate modifications) or replaced with another base without significant effect. Helix 4 can be formed from two separate molecules, i.e., without a connecting loop. The connecting loop when present may be a ribonucleotide with or without modifications to its base, sugar or phosphate. “q” is≧2 bases. The connecting loop can also be replaced with a non-nucleotide linker molecule. H refers to bases A, U, or C. Y refers to pyrimidine bases. “______” refers to a covalent bond. (Burke, et al., 1996, Nucleic Acids & Mol. Biol., 10, 129; Chowrira, et al., U.S. Pat. No. 5,631,359).

FIG. 2 is a diagram of a hammerhead ribozyme targeted against VEGF-receptor RNA (VEGF-R-1 ribozyme). The ribozyme has a 4 base pair stem II, four phosphorothioate linkages at the 5′-end, a 2′-C-allyl substitution at position 4, ribonucleotides at five positions, 2′-O-methyl substitution at the remaining positions and an inverted abasic nucleotide substitution at the 3′-end.

FIG. 3 illustrates the concentrations of ribozyme in retina and capsule of hyperoxic treated neonatal mice after intravitreal administration of 5 μg free or formulated VEGF-R-1 ribozyme (supplemented with 10×10⁶ cpm ³²P VEGF-R-1 ribozyme) formulated in an EPC:DOTAP:PEG liposome or non-formulated. Mice are administered ribozyme either immediately upon their removal from the hyperoxic chamber or five days after their removal from the hyperoxic chamber.

FIG. 4 illustrates the percent of intact ribozyme in the retina and capsule of hyperoxic neonatal mice after intravitreal administration of 5 μg free or formulated VEGF-R-1 ribozyme (supplemented 10×10⁶ cpm ³²P VEGF-R-1 ribozyme). Mice were administered ribozyme either immediately upon their removal from the hyperoxic chamber or five days after their removal from the hyperoxic chamber.

FIG. 5 illustrates the plasma concentrations of ribozyme in hyperoxic treated neonatal mice after intravitreal administration of 5 μg free or formulated VEGF-R-1 ribozyme (supplemented with 10×10⁶ cpm ³²P VEGF-R-1 ribozyme) formulated in an EYPC:DOTAP:PEG liposome or non-formulated (EYPC=egg yolk phosphatidylcholine=EPC). Mice were administered ribozyme either immediately upon their removal from the hyperoxic chamber of five days after their removal from the hyperoxic chamber.

FIG. 6 illustrates the percent of intact ribozyme in plasma of hyperoxic neonatal after intravitreal administration of 5 μg free or formulated VEGF-R-1 ribozyme (supplemented with 10×10⁶ cpm ³²P VEGF-R-1 ribozyme). Mice were administered ribozyme either immediately upon their removal from the hyperoxic chamber or five days after their removal from the hyperoxic chamber.

FIG. 7 illustrates the liver and kidney concentrations of ribozyme in hyperoxic treated neonatal mice after intravitreal administration of 5 μg free or formulated VEGF-R ribozyme (supplemented with 10×10⁶ cpm ³²P VEGF-R ribozyme) formulated in an EPC:DOTAP:PEG liposome or non-formulated. Mice were administered ribozyme either immediately upon their removal from the hyperoxic chamber or five days after their removal from the hyperoxic chamber

FIG. 8 illustrates the percent of intact ribozyme in liver and kidney of hyperoxic neonatal mice after intravitreal administration of 5 μg free or formulated VEGF-R-1 ribozyme (supplemented with 10×10⁶ cpm ³²P VEGF-R-1). Mice were administered ribozyme either immediately upon their removal from the hyperoxic chamber or five days after their removal from the hyperoxic chamber.

FIG. 9 illustrates the plasma levels for different liposomal ribozyme formulations in the murine Lewis lung model. Curves are normalized to 1 mg/kg ribozyme dose, although actual doses varied somewhat, depending on the efficiency of ribozyme encapsulation. Each animal received a constant lipid dose (3 μmol). SM=sphingomyelin.

FIG. 10 illustrates the plasma levels of intact ribozyme for three different types of liposome formulations as indicated.

FIG. 11 illustrates the time course for ribozyme exposure in primary tumors following a single intravenous administration. Liposome 1=EPC/DODAC/Chol/PEG-CerC20; Liposome 2=EPC/DODAC/Chol/PEG-CerC14.

FIG. 12 illustrates the elimination profiles for lipid ([³H]-CHE) and ribozyme ([³²P-CHE) tracers using three different types of liposomes. Top=plasma levels; Bottom=tumor levels.

FIG. 13 illustrates the decrease in tumor growth in the Lewis Lung Carcinoma Model after treatment with liposome encapsulated formulated VEGF-R-1 ribozyme.

FIG. 14 illustrates the stability of ribozyme formulation after delivery to the tumor. The stability was measured by measuring the percent of full length ribozyme compared to total isolated radioactivity following PAGE analysis.

### DETAILED DESCRIPTION OF THE INVENTION AND PREFERRED EMBODIMENTS

### I. Glossary

A. Abbreviations and Definitions

The following abbreviations are used herein: CHO, Chinese hamster ovary cell line; B16, murine melanoma cell line; DC-Chol, 3β-(N-(N′,N′-dimethylaminoethane)carbamoyl)cholesterol (see, Gao, et al., Biochem. Biophys. Res. Comm., 179:280-285 (1991)); DDAB, N,N-distearyl-N,N-dimethylammonium bromide; DMRIE, N-(1,2-dimyristyloxyprop-3-yl)-N,N-dimethyl-N-hydroxyethyl ammonium bromide; DODAC, N,N-dioleyl-N,N-dimethylammonium chloride (see commonly owned patent application U.S. Ser. No. 08/316,399, incorporated herein by reference); DOGS, diheptadecylamidoglycyl spermidine; DOPE, 1,2-sn-dioleoylphoshatidylethanolamine; DOSPA, N-(1-(2,3-dioleyloxy)propyl)-N-(2-(sperminecarboxamido)ethyl)-N,N-dimethylammonium trifluoroacetate; DOTAP, N-(1-(2,3-dioleoyloxy)propyl)-N,N,N-trimethylammonium chloride; DOTMA, N-(1-(2,3-dioleyloxy)propyl)-N,N,N-trimethylammonium chloride; ESM, egg sphingomyelin; RT, room temperature; TBE, Tris-Borate-EDTA (89 mM in Tris-borate and 2 mM in EDTA); HEPES, 4-(2-hydroxyethyl)-1-piperazineethanesulfonic acid; PBS, phosphate-buffered saline; EGTA, ethylenebis(oxyethylenenitrilo)-tetraacetic acid.

The term “acyl” refers to a radical produced from an organic acid by removal of the hydroxyl group. Examples of acyl radicals include acetyl, pentanoyl, palmitoyl, stearoyl, myristoyl, caproyl and oleoyl.

As used herein, the term “pharmaceutically acceptable anion” refers to anions of organic and inorganic acids which provide non-toxic salts in pharmaceutical preparations. Examples of such anions include chloride, bromide, sulfate, phosphate, acetate, benzoate, citrate, glutamate, and lactate. The preparation of pharmaceutically acceptable salts is described in Berge, et al., J. Pharm. Sci., 66:1-19 (1977), incorporated herein by reference.

The term “lipid” refers to any suitable material resulting in a bilayer such that a hydrophobic portion of the lipid material orients toward the bilayer while a hydrophilic portion orients toward the aqueous phase. Amphipathic lipids are necessary as the primary lipid vesicle structural element. Hydrophilic characteristics derive from the presence of phosphate, carboxylic, sulfato, amino, sulfhydryl, nitro, and other like groups. Hydrophobicity could be conferred by the inclusion of groups that include, but are not limited to, long chain saturated and unsaturated aliphatic hydrocarbon groups and such groups substituted by one or more aromatic, cycloaliphatic or heterocyclic group(s). The preferred amphipathic compounds are phosphoglycerides and sphingolipids, representative examples of which include phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine or dilinoleoylphosphatidylcholine could be used. Other compounds lacking in phosphorus, such as sphingolipid and glycosphingolipid families are also within the group designated as lipid. Additionally, the amphipathic lipids described above may be mixed with other lipids including triglycerides and sterols.

The term “neutral lipid” refers to any of a number of lipid species which exist either in an uncharged or neutral zwitterionic form at physiological pH. Such lipids include, for example diacylphosphatidylcholine, diacylphosphatidylethanolamine, ceramide, sphingomyelin, cephalin, and cerebrosides.

The term “non-cationic lipid” refers to any neutral lipid as described above as well as anionic lipids. Examples of anionic lipids include cardiolipin, diacylphosphatidylserine and diacylphosphatidic acid.

The term “cationic lipid” refers to any of a number of lipid species which carry a net positive charge at physiological pH. Such lipids include, but are not limited to, DODAC, DOTMA, DDAB, DOTAP, DC-Chol and DMRIE. Additionally, a number of commercial preparations of cationic lipids are available which can be used in the present invention. These include, for example, LIPOFECTIN® (commercially available cationic liposomes comprising DOTMA and DOPE, from GIBCO/BRL, Grand Island, N.Y., USA); LIPOFECTAMINE® (commercially available cationic liposomes comprising DOSPA and DOPE, from GIBCO/BRL); and TRANSFECTAM® (commercially available cationic lipids comprising DOGS in ethanol from Promega Corp., Madison, Wis., USA).

The term “nucleic acid catalyst” or, alternatively, “enzymatic nucleic acid molecules” is used herein to refer to a nucleic acid molecule capable of catalyzing (i.e., altering the velocity and/or rate of) a variety of reactions including the ability to repeatedly cleave other separate nucleic acid molecules (endonuclease activity) in a nucleotide base sequence-specific manner. Such a molecule with endonuclease activity may have complementarity in a substrate binding region to a specified gene target, and also has enzymatic activity that specifically cleaves RNA or DNA in that target. That is, the nucleic acid molecule with endonuclease activity is able to intramolecularly or intermolecularly cleave RNA or DNA and thereby inactivate a target RNA or DNA molecule. This complementarity functions to allow sufficient hybridization of the enzymatic RNA molecule to the target RNA or DNA to allow the cleavage to occur. 100% complementarity is preferred, but complementarity as low as 50-75% may also be useful in this invention. The nucleic acids may be modified at the base and/or phosphate groups. The term enzymatic nucleic acid is used interchangeably with the following phrases: ribozymes, catalytic RNA, enzymatic RNA, catalytic DNA, catalytic oligonucleotides, nucleozyme, DNAzyme, RNA enzyme, endoribonuclease, endonuclease, minizyme, leadzyme, oligozyme or DNA enzyme. All of these terms describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving activity to the molecule.

By “enzymatic portion” or “catalytic domain” is meant that portion/region of the ribozyme essential for cleavage of a nucleic acid substrate.

By “substrate binding arm” or “substrate binding domain” is meant that portion/region of a ribozyme which is complementary to (i.e., able to base-pair with) a portion of its substrate. Generally, such complementarity is 100%, but can be less if desired. For example, as few as 10 bases out of 14 may be base-paired. That is, the arms of the ribozymes contain sequences within a ribozyme which are intended to bring ribozyme and target together through complementary base-pairing interactions. The ribozyme of the invention may have binding arms that are contiguous or non-contiguous and may be varying lengths. The length of the binding arm(s) are preferably greater than or equal to four nucleotides; specifically 12-100 nucleotides; more specifically 14-24 nucleotides long. If a ribozyme with two binding arms are chosen, then the length of the binding arms are symmetrical (i.e., each of the binding arms is of the same length; e.g., six and six nucleotides or seven and seven nucleotides long) or asymmetrical (i.e., the binding arms are of different length; e.g., six and three nucleotides or three and six nucleotides long).

By “nucleic acid molecule” as used herein is meant a molecule having nucleotides. The nucleic acid can be single, double or multiple stranded and may comprise modified or unmodified nucleotides or non-nucleotides or various mixtures and combinations thereof. An example of a nucleic acid molecule according to the invention is a gene which encodes for macromolecule such as a protein.

By “complementarity” as used herein is meant a nucleic acid that can form hydrogen bond(s) with other nucleic acid sequence by either traditional Watson-Crick or other non-traditional types (for example, Hoogsteen type) of base-paired interactions.

The term “transfection” as used herein, refers to the introduction of polyanionic materials, particularly nucleic acids, into cells. The term “lipofection” refers to the introduction of such materials using liposome complexes. The polyanionic materials can be in the form of DNA or RNA which is linked to expression vectors to facilitate gene expression after entry into the cell. Thus the polyanionic material used in the present invention is meant to include DNA having coding sequences for structural proteins, receptors and hormones, as well as transcriptional and translational regulatory elements (i.e., promoters, enhancers, terminators and signal sequences) and vector sequences. Methods of incorporating particular nucleic acids into expression vectors are well known to those of skill in the art, but are described in detail in, for example, Sambrook, et al., Molecular Cloning: A Laboratory Manual (2nd ed.), Vols. 1-3, Cold Spring Harbor Laboratory, (1989) or Current Protocols in Molecular Biology, F. Ausubel, et al., ed. Greene Publishing and Wiley-Interscience, New York (1987), both of which are incorporated herein by reference.

“Expression vectors”, “cloning vectors”, or “vectors” are often plasmids or other nucleic acid molecules that are able to replicate in a chosen host cell. Expression vectors may replicate autonomously, or they may replicate by being inserted into the genome of the host cell, by methods well known in the art. Vectors that replicate autonomously will have an origin of replication or autonomous replicating sequence (ARS) that is functional in the chosen host cell(s). Often, it is desirable for a vector to be usable in more than one host cell, e.g., in E. coli for cloning and construction, and in a mammalian cell for expression.

The term “biological system,” as used herein, includes reference to a eukaryotic system or a prokaryotic system, and can be a bacterial cell, a plant cell or a mammalian cell, and can be of plant origin, mammalian origin, yeast origin, Drosophila origin, or archebacterial origin.

The term “PEG-Ceramide” or, interchangeably, “PEG-Cer” is used herein to refer to a compound or conjugate wherein polyethylene glycol is covalently linked to a ceramide molecule as described for example by Choi, et al., 1996, supra (incorporated by reference herein).

### II. General

The present invention provides compositions and methods for delivering nucleic acid catalysts, i.e., enzymatic nucleic acid moleucles, to a biological system. More particularly, the present invention provides compositions for delivering nucleic acid catalysts to a cell, the composition comprising a lipid, a polyethyleneglycol-ceramide (PEG-Cer) conjugate and a nucleic acid catalyst (e.g., a VEGF-R-1 ribozyme). In a presently preferred embodiment, the composition comprises a non-cationic lipid, a cationic lipid, a polyethyleneglycol-ceramide (PEG-Cer) conjugate and a nucleic acid catalyst. Such compositions have improved circulation characteristics and serum-stability and, thus, can be used to deliver nucleic acid catalysts to cells both in vitro and in vivo, and in the presence or absence of serum.

As noted above, in one embodiment, the compositions of the present invention comprise, inter alia, a lipid, a PEG-Cer conjugate and a nucleic acid catalyst. As explained hereinbelow, numerous lipids can be used in the compositions of the present invention. In preferred embodiments, the lipid is a diacylphosphatidylcholine and, in particular, egg yolk phosphatidylcholine. In addition, the compositions of the present invention comprise a cationic lipid. As explained hereinbelow, numerous cationic lipids can be used in the compositions of the present invention. In preferred embodiments, the cationic lipid is N,N-dioleyl-N,N-dimethylammonium chloride (DODAC) or 1,2-dioleoyloxy-3-(N,N,N-trimethylamino)propane chloride (DOTAP). In addition, the compositions of the present invention contain a PEG-Cer conjugate having fatty acid groups of various chain lengths. Preferably, the ceramide has a fatty acid group having between 6 and 24 carbon atoms. In a preferred embodiment, the compositions of the present invention comprise, inter alia, a non-cationic lipid (e.g., a diacylphosphatidylcholine), a cationic lipid (e.g., DODAC, DOTAP, etc.), a PEG-Cer conjugate and a nucleic acid catalyst.

The non-cationic lipids used in the present invention can be any of a variety of neutral uncharged, zwitterionic or anionic lipids. Examples of neutral lipids which are useful in the present methods are diacylphosphatidylcholines, diacylphosphatidylethanolamines, ceramides, sphingomyelins, cephalins and cerebrosides. Other lipids, such as lysophosphatidylcholine and lysophosphatidylethanolamine, can also be present. In preferred embodiments, the non-cationic lipids are diacylphosphatidylcholines (e.g., dioleoylphosphatidylcholine, dipalmitoylphosphatidylcholine and dilinoleoylphosphatidylcholine), diacylphosphatidylethanolamine (e.g., dioleoylphosphatidylethanolamine and palmitoyloleoylphosphatidylethanolamine), ceramide or sphingomyelin. The acyl groups in these lipids are preferably acyl groups derived from fatty acids having C₁₀-C₂₄ carbon chains. More preferably, the acyl groups are lauroyl, myristoyl, palmitoyl, stearoyl or oleoyl. In particularly preferred embodiments, the non-cationic lipid will be a diacylphosphatidylcholine and, in particular, egg yolk phosphatidylcholine. Other non-cationic lipids known to and used by those of skill in the art can be used in the compositions of the present invention.

Examples of suitable cationic lipids include, but are not limited to, the following: DC-Chol, 3β(N-(N′,N′-dimethylaminoethane)carbamoyl)cholesterol (see, Gao, et al., Biochem. Biophys. Res. Comm, 179:280-285 (1991); DDAB, N,N-distearyl-N,N-dimethylammonium bromide; DMRIE, N-(1,2-dimyristyloxyprop-3-yl)-N,N-dimethyl-N-hydroxyethyl ammonium bromide; DODAC, N,N-dioleyl-N,N-dimethylammonium chloride (see, commonly owned U.S. patent application Ser. No. 08/316,399, filed Sep. 30, 1994, which is incorporated herein by reference); DOGS, diheptadecylamidoglycyl spermidine; DOSPA, N-(1-(2,3-dioleyloxy)propyl)-N-(2-(sperminecarboxamido)ethyl)-N,N-dimethylammonium trifluoroacetate; DOTAP, N-(1-(2,3-dioleoyloxy)propyl)-N,N,N-trimethylammonium chloride; DOTMA, N-(1-(2,3-dioleyloxy)propyl)-N,N,N-trimethylammonium chloride; LIPOFECTIN, a commercially available cationic lipid comprising DOTMA and DOPE (GIBCO/BRL, Grand Island, N.Y.) (U.S. Pat. Nos. 4,897,355; 4,946,787; and 5,208,036 issued to Epstein, et al.); LIPOFECTACE or DDAB (dimethyldioctadecyl ammonium bromide) (U.S. Pat. No. 5,279,883 issued to Rose); LIPOFECTAMINE, a commercially available cationic lipid composed of DOSPA and DOPE (GIBCO/BRL, Grand Island, N.Y.); TRANSFECTAM, a commercially available cationic lipid comprising DOGS (Promega Corp., Madison, Wis.). In a presently preferred embodiment, the cationic lipid is N,N-dioleyl-N,N-dimethylammonium chloride (DODAC) or 1,2-dioleoyloxy-3-(N,N,N-trimethylamino)propane chloride (DOTAP).

In addition to the non-cationic and cationic lipids, the compositions of the present invention contain a PEG-Cer conjugate having fatty acid groups of various chain lengths. Preferably, the ceramide has a fatty acid group having between 6 and 24 carbon atoms. In particularly preferred embodiments, the PEG-Cer conjugate has fatty acid groups comprising 8, 14, or 20 carbon atoms, designated as PEG-Cer-C8 (or PEG-C8), PEG-Cer-C14 (or PEG-C14); and PEG-Cer-C20 (or PEG-C20), respectively. Methods suitable for synthesizing such PEG-Cer conjugates are disclosed in Choi, et al., PCT Publication No. WO 96/10391 and Holland, et al., PCT Publication No. WO 96/10392, the teachings of both of which are incorporated herein by reference.

The lipid and PEG-Cer conjugate are combined in various proportions which allow for the effective delivery of nucleic acid catalysts to a desired cell or biological system of interest. In a preferred embodiment, the non-cationic lipid, the cationic lipid and the PEG-Cer conjugate are combined in various proportions which allow for the effective delivery of nucleic acid catalysts to a desired cell or biological system of interest. Typically, the non-cationic lipid is present at a concentration ranging from about 20 mole percent to about 95 mole percent. More preferably, the non-cationic lipid is present at a concentration ranging from about 40 mole percent to about 60 mole percent. More preferably, the non-cationic lipid is present at a concentration of about 50 mole percent. The cationic lipid is typically present at a concentration ranging from about 5 mole percent to about 80 mole percent. More preferably, the cationic lipid is present at a concentration ranging from about 10 mole percent to about 40 mole percent. More preferably, the cationic lipid is present at a concentration of about 15 mole percent. The PEG-Cer conjugate is typically present at a concentration ranging from about 0.5 mole percent to about 50 mole percent. More preferably, the PEG-Cer conjugate is present at a concentration ranging from about 5 mole percent to about 20 mole percent. More preferably, the PEG-Cer conjugate is present at a concentration of about 10 mole percent.

In a presently preferred embodiment, the compositions of the present invention also contain cholesterol. Cholesterol can be added, for example, to increase the thermal transition temperature of the composition, for example, in cases where it is necessary to increase the stability of the composition in a biological system and/or to reduce the rate of leakage of encapsulated enzymatic nucleic acid. Cholesterol, if included, is generally present at a concentration ranging from 0.02 mole percent to about 50 mole percent, more preferably, at a concentration ranging from about 15 mole percent to about 45 mole percent and, more preferably, at a concentration of about 25 mole percent.

In addition to the foregoing, the compositions of the present invention can further include additional components. For instance, the compositions can contain additional lipids, such as a pH-sensitive lipid, which may be added to increase the amount of nucleic acid catalysts (e.g., VEGF-R-1 ribozyme) that can be encapsulated in the formulation.

The enzymatic nucleic acid molecules of the invention are added as a composition as described herein. As explained herein, the nucleic acid catalyst:PEG-Cer compositions can be locally administered to relevant tissues through the use of a catheter, or infusion pump. Using the methods described herein, other enzymatic nucleic acid molecules that cleave target nucleic acid can be derived and used as described herein. Specific examples of nucleic acid catalysts of the instant invention are provided below in the Figures and Examples (See, e.g., Example 7).

Such enzymatic nucleic acid molecules can be delivered exogenously to specific cells as required. In the preferred hammerhead motif, the small size (less than 60 nucleotides, preferably between 30-40 nucleotides in length) of the molecule allows the cost of treatinent io be reduced.

### III. Formulation Methods:

The PEG-Cer formulated nucleic acid catalyst compositions of the present invention can using a variety of different approaches known in the art (see, e.g., Liposomes, A Practical Approach. 1997. Ed. R. R. C. IRL Press; Lipsome Technology, 1993. Ed. Gregoriadis, G., CRC Press; Szoka, et al., 1980, Ann. Rev. Biophys. Bioeng., 9:467; all of these are incorporated by reference herein). In addition, other efficient and rapid methods have now been developed for formulating ribozymes with lipid-based carriers that are suitable for the cellular delivery of ribozymes.

A. Reverse Phase Evaporation

The desired lipid-PEG-Cer containing composition is mixed together, solubilized in chloroform and dried into a film. The composition is then resuspended in a suitable organic solvent (e.g., diether or isopropyl ether). To this mixture, the nucleic acdi catalyst (e.g., a VEGF-R-1 ribozyme) to be encapsulated is added in a 1:3 ratio with solvent. The mixture is then sonicated to form an emulsion. This is thought to cause formation of inverted micelles, with hydrophilic head groups solubilized in the aqueous droplets of the emulsion.

As the solvent is evaporated, for example, under vacuum, the inverted micelles are forced into closer proximity creating a gel-like substance. After a minimum quantity of solvent is removed, the inverted micelles spontaneously invert to bilayers (in Lipsome Technology, 1993. Ed. Gregoriadis, G. CRC press). This protocol essentially builds the liposome around the water droplet. Like the detergent dialysis method, infra, a cationic amphiphile is used herein to increase entrapment of the VEGF-R-1 ribozyme in the liposome composition. Encapsulation efficiencies vary depending on lipid composition, solvent evaporation times and solute concentrations, but generally are greater than those seen with passive encapsulation.

B. Passive Encapsulation and Extrusion Methods

The desired lipid-PEG-Cer containing composition is mixed together, solubilized in an organic solvent and dried into a lipid film. By adding aqueous phase buffer to this film, the lipids spontaneously form vesicles due to hydrophobic interactions of the lipid fatty acid chains. Because of the amphipathic nature of the lipids, they will assemble to form aggregates with hydrophobic interiors and hydrophilic exteriors. This process results in the formation of Multilamellar vesicles (MLV's) which are comprised of a series of concentric spheres with aqueous lumen between the bilayers. Quickly freezing the dispersion in liquid nitrogen and thawing to above the phase transitional temperature (Tₘ) of the lipid mixture may increase the trapping efficiency by bringing the transmembrane solute concentration to equilibrium (Alino, et al., 1990, J. Microencapsulation, 7:497-503, incorporated by reference herein).

Since this protocol generates MLV's which are in the micron range, they are usually unsuitable for systemic administration. In order to reduce liposomal diameters, they are forced through polycarbonate filters of defined pore size (0.1 mm) using inert gas (e.g., nitrogen) in a device known as an Extruder™ (Lipex Biomembranes, Vancouver, B.C.). This procedure is the easiest protocol for liposomal formation. However, since the solute is passively captured within the liposome, entrapment efficiencies are very low and dependent on geometric constraints of the vesicles.

C. Dialysis Method

As above, the lipid combinations are solubilized in an organic solvent, together and dried into a film. The formulation is then solubilized in an aqueous buffer containing a suitable detergent (e.g., n-octyl-D-glucopyranoside, sodium cholate) and the nucleic acid catalyst (e.g., VEGF-R-1 ribozyme) to be encapsulated. The detergent interacts with the lipids and minimizes the interaction between the hydrophobic portion of the amphiphiles and water by forming micelles (in Liposomes, A Practical Approach. 1997. Ed. R. R. C. IRL Press). Sufficient detergent should be added so that all of the lipid bilayers are converted into detergent-lipid mixed micelles.

The detergent is then slowly removed, usually by passive diffusion dialysis tubing. As the detergent is slowly removed, the lipids form unilamellar vesicles which will encapsulate the ribozymes.

Detergent dialysis generally results in higher trapping efficiencies compared to passive encapsulation and can lessen the amount of extrusion necessary since smaller vesicles are formed using this method (nanometer range). Trapping efficiencies can be increased by using charged amphiphiles, such as cationic lipids, which may be used to associate with charged solutes (e.g., cationic lipid with ribozymes).

D. Bligh & Dyer Extraction

Hydrophobic cationic lipid, hydrophilic nucleic acid catalysts and other lipids are all solubilized in a solution of CHCl₃, Methanol and Water (1:2.1:1). Excess chloroform and water are then added to separate the organic and aqueous phases. At the organic/aqueous interphase the cationic lipid ion-pairs with the ribozyme, increasing the hydrophobicity of the solute. The complex becomes solubilized in chloroform and migrates into the organic phase.

The aqueous phase is then removed and the organic phase is dried down to remove all of the chloroform. The lipid/solute film is then hydrated in an aqueous buffer. Encapsulation is usually quantitative as long as a minimum charge ratio between cationic lipid and ribozyme exists. The minimum charge ratio generally varies for different cationic lipids.

### IV. The Nucleic Acid Catalysts: Design, Synthesis, Deprotection and Purification

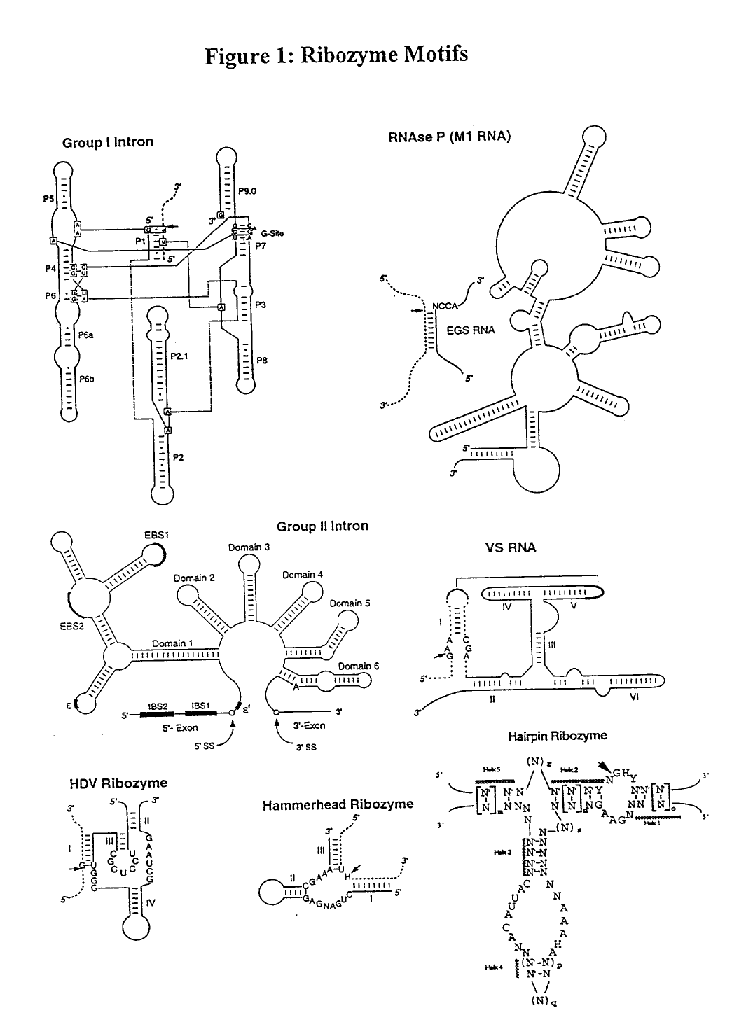

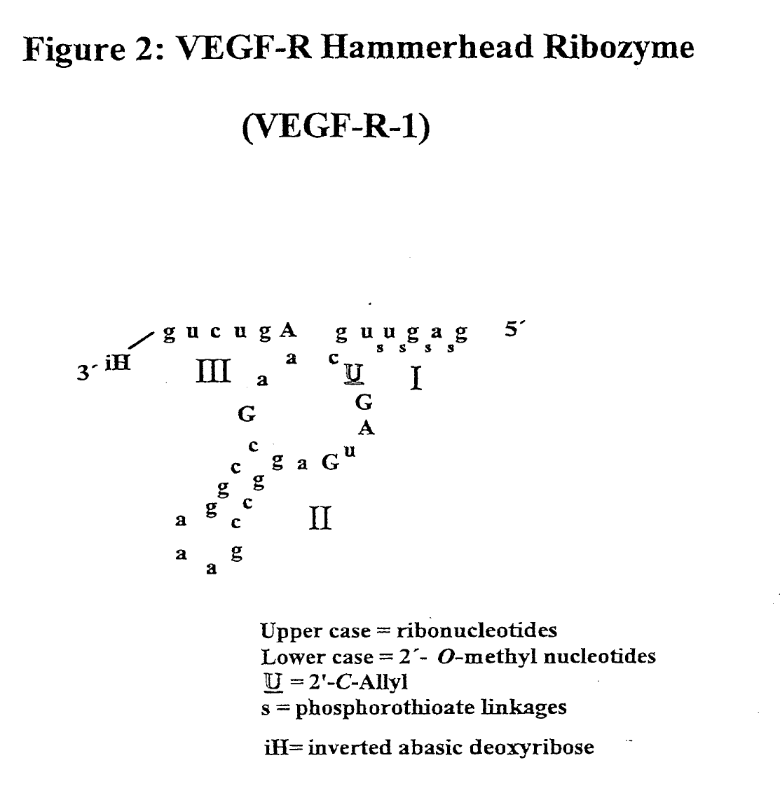

In one aspect enzymatic nucleic acid molecule is formed in a hammerhead (see, e.g., FIGS. 1 and 2) or a hairpin motif (see, FIG. 1), but may also be formed in the motif of a hepatitis delta virus (HDV), group 1 intron, RNaseP RNA (in association with an eternal guide sequence) or Neurospora VS RNA (see, FIG. 1). Examples of such hammerhead motifs are described by Rossi, et al., 1992, Aids Research and Human Retroviruses 8, 183; Usman, et al., 1996, Curr. Op. Struct. Biol., 1, 527; of hairpin motifs by Hampel, et al., EP 0360257; Hampel and Tritz, 1989, Biochemistry 28, 4929; and Hampel, et al., 1990, Nucleic Acids Res. 18, 299; Chowrira, et al., U.S. Pat. No. 5,631,359; an example of the hepatitis delta virus motif is described by Perrotta and Been, 1992 Biochemistry, 31, 16; Been, et al., U.S. Pat. No. 5,625,047; of the RNaseP motif by Guerrier-Takata, et al., 1983, Cell 35, 849; Forster and Altman, 1990, Science 249, 783; Neurospora VS RNA ribozyme motif is described by Collins (Saville and Collins, 1990 Cell 61, 685-696; Saville and Collins, 1991 Proc. Natl. Acad. Sci. USA 88, 8826-8830; Guo and Collins, 1995 EAMBO J. 14, 368) and of the Group I intron by Zaug, et al., 1986, Nature, 324, 429; Cech et al., U.S. Pat. No. 4,987,071. These specific motifs are not limiting in the invention and those skilied in the art will recognize that all that is important in an enyzmatic nucleic acid molecule with endonuclease activity of this invention is that it has a specific substrate binding site which is complementary to one or more of the target gene RNA and that it have nucleotide sequences within or surrounding that substrate binding site which impart an RNA cleaving activity to the molecule. The length of the binding site varies for different ribozyme motifs, and a person skilled in the art will recognize that to achieve an optimal ribozyme activity the length of the binding arm should be of sufficient length to form a stable interaction with the target nucleic acid sequence.

The enzymatic nucleic acid molecules of the instant invention can be expressed within cells from eukaryotic promoters (e.g., Izant and Weintraub, 1985, Science, 229:345; McGarry and Lindquist, 1986, Proc. Natl. Acad. Sci. USA, 83:399; Scanlon, et al., 1991, Proc. Natl. Acad. Sci. USA, 88:10591-5; Kashani-Sabet, et al., 1992, Antisense Res. Dev., 2:3-15; Dropulic, et al., 1992, J. Virol., 66:1432-41; Weerasinghe, et al., 1991, J. Virol., 65:5531-4; Ojwang, et al., 1992, Proc. Natl. Acad. Sci. USA, 89:10802-6; Chen, et al., 1992, Nucleic Acids Res., 20:4581-9; Sarver, et al., 1990, Science, 247:1222-1225; Thompson, et al., 1995, Nucleic Acids Res., 23:2259; Good, et al., 1997, Gene Therapy, 4:45; all of the references are hereby incorporated in their totality by reference herein). Those skilled in the art realize that any nucleic acid can be expressed in eukaryotic cells from the appropriate DNA/RNA vector. The activity of such nucleic acids can be augmented by their release from the primary transcript by a ribozyme (Draper, et al., PCT WO 93/23569, and Sullivan, et al., PCT WO 94/02595; Ohkawa, et al., 1992, Nucleic Acids Symp. Ser., 27:15-6; Taira, et al., 1991, Nucleic Acids Res., 19:5125-30; Ventura, et al., 1993, Nucleic Acids Res., 21:3249-55, Chowrira, et al., 1994, J. Biol. Chem., 269:25856; all of the references are hereby incorporated in their totality by reference herein).

By “vectors” is meant any nucleic acid- and/or viral-based technique used to render active a desired nucleic acid (see, above).

In another aspect of the invention, enzymatic nucleic acid molecules that cleave target molecules are expressed from transcription units (for a review, see, Couture and Stinchcomb, 1996, TIG, 12:510, the teachings of which are incorporated by reference herein).

The nucleic acid catalysts used in the compositions and methods of the present invention can be made using the method of synthesis of enzymatic nucleic acid molecules as described in Usman, et al., 1987. J. Am. Chem. Soc., 109:7845; Scaringe, et al., 1990, Nucleic Acids Res., 18:5433; and Wincott, et al., 1995, Nucleic Acids Res., 23:2677-2684, and makes use of common nucleic acid protecting and coupling groups, such as dimethoxytrityl at the 5′-end, and phosphoramidites at the 3′-end. Small scale synthesis were conducted on a 394 Applied Biosystems, Inc. synthesizer using a modified 2.5 μmol scale protocol with a 5 min coupling step for alkylsilyl protected nucleotides and 2.5 min coupling step for 2′-O-methylated nucleotides. Table I outlines the amounts, and the contact times of the reagents used in the synthesis cycle. A 6.5-fold excess (163 μL of 0.1 M=16.3 μmol) of phosphoramidite and a 24-fold excess of S-ethyl tetrazole (238 μL of 0.25 M=59.5 μmol) relative to polymer-bound 5′-hydroxyl is used in each coupling cycle. Average coupling yields on the 394 Applied Biosystems, Inc. synthesizer, determined by calorimetric quantitation of the trityl fractions, is 97.5-99%. Other oligonucleotide synthesis reagents for the 394 Applied Biosystems, Inc. synthesizer: detritylation solution was 2% TCA in methylene chloride (ABI); capping was performed with 16% N-methyl imidazole in THF (ABI) and 10% acetic anhydride/10% 2,6-lutidine in THF (ADI); oxidation solution was 16.9 mM I₂, 49 mM pyridine, 9% water in THF (Millipore). B & J Synthesis Grade acetonitrile is used directly from the reagent bottle. S-Ethyl tetrazole solution (0.25 M in acetonitrile) is made up from the solid obtained from American International Chemical, Inc.

TABLE I
2.5 μmol RNA Synthesis Cycle

| | | | | Wait |
|---|---|---|---|---|
| Reagent | Equivalents | Amount | | Time* |
| Phosphoramidites | 6.5 | 163 | μL | 2.5 |
| S-Ethyl Tetrazole | 23.8 | 238 | μL | 2.5 |
| Acetic Anhydride | 100 | 233 | μL | 5 sec |
| N-Methyl Imidazole | 186 | 233 | μL | 5 sec |
| TCA | 83.2 | 1.73 | mL | 21 sec |
| Iodine | 8.0 | 1.18 | mL | 45 sec |
| Acetonitrile | NA | 6.67 | mL | NA |

*Wait time does not include contact time during delivery.

Deprotection of the chemically synthesized nucleic acid catalysts of the invention is performed as follows. The polymer-bound oligoribonucleotide, trityl-off, is transferred from the synthesis column to a 4 mL glass screw top vial and suspended in a solution of methylamine (MA) at 65° C. for 10 min. After cooling to −20° C., the supernatant is removed from the polymer support. The support is washed three times with 1.0 mL of EtOH:MeCN:H₂O/3:1:1, vortexed and the supernatant is then added to the first supernatant. The combined supernatants, containing the oligoribonucleotide, are dried to a white powder.

The base-deprotected oligoribonucleotide is resuspended in anhydrous TEA-HF/NMP solution (250 μL of a solution of 1.5 mL N-methylpyrrolidinone, 750 μL TEA and 1.0 mL TEA-3HF to provide a 1.4M HF concentration) and heated to 65° C. for 1.5 h. The resulting, fully deprotected oligomer is quenched with 50 mM TEAB (9 mL) prior to anion exchange desalting.

For anion exchange desalting of the deprotected oligomer, the TEAB solution is loaded on to a Qiagen 500® anion exchange cartridge (Qiagen Inc.) that is prewashed with 50 mM TEAB (10 mL). After washing the loaded cartridge with 50 mM TEAB (10 mL), the RNA is eluted with 2 M TEAB (10 mL) and dried down to a white powder. The average stepwise coupling yields are generally>98% (Wincott, et al., 1995, Nucleic Acids Res., 23:2677-2684).

The ribozymes of the instant invention can also be synthesized from DNA templates using bacteriophage T7 RNA polymerase (Milligan and Uhlenbeck, 1989, Methods Enzymol., 180:51).

Once synthesized, the nucleic acid catalysts of the present invention are purified by gel electrophoresis using general methods or are purified by high pressure liquid chromatography (HPLC, see, Wincott, et al., supra) the totality of which is hereby incorporated herein by reference) and are resuspended in water.

By “nucleotide” as used herein is as recognized in the art to include natural bases (standard), and modified bases well known in the art. Such bases are generally located at the 1′ position of a sugar moiety. Nucleotide generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, for example, Usman and McSwiggen, supra, Eckstein, et al., International PCT Publication No. WO 92/07065, Usman, et al., International PCT Publication No. WO 93/15187; all hereby incorporated by reference herein). There are several examples of modified nucleic acid bases known in the art and has recently been summarized by Limbach, et al., 1994, Nucleic Acids Res., 22:2183. Some of the non-limiting examples of base modifications that can be introduced into enzymatic nucleic acids without significantly effecting their catalytic activity include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g., 6-methyluridine) and others (Burgin, et al., 1996, Biochemistry, 35:14090). By “modified bases” in this aspect is meant nucleotide bases other than adenine, guanine, cytosine and uracil at 1′ position or their equivalents; such bases may be used within the catalytic core of the enzyme and/or in the substrate-binding regions.

The catalytic activity of the nucleic acid catalysts described in the instant invention can be optimized as described by Draper, et al., supra. The details will not be repeated here, but include altering the length of the ribozyme binding arms, or chemically synthesizing the ribozymes with modifications (base, sugar and/or phosphate) that prevent their degradation by serum ribonucleases and/or enhance their enzymatic activity (see, e.g., Eckin, et al., International Publication No. WO 92/07065; Perrault, et al., 1990, Nature, 344:565; Pieken, et al., 1991, Science, 253:314; Usman and Cedergren, 1992, Trends in Biochem. Sci., 17:334; Usman, et al., International Publication No. WO 93/15187; and Rossi, et al., International Publication No. WO 91/03162; Sproat, U.S. Pat. No. 5,334,711; and Burgin, et al., supra; all of these describe various chemical modifications that can be made to the base, phosphate and/or sugar moieties of enzymatic RNA molecules). Modifications which enhance their efficacy in cells, and removal of bases from stem loop structures to shorten RNA synthesis times and reduce chemical requirements are desired. (All these publications are hereby incorporated by reference herein).

There are several examples in the art describing sugar and phosphate modifications that can be introduced into the enzymatic nucleic acid molecules without significantly effecting catalysis and with significant enhancement in their nuclease stability and efficacy. Ribozymes are modified to enhance stability and/or enhance catalytic activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS, 17:34; Usman, et al., 1994, Nucleic Acids Symp. Ser., 31:163; Burgin, et al., 1996, Biochemistry, 35:14090). Sugar modification of enzymatic nucleic acid molecules have been extensively described in the art (see, Eckstein, et al., International Publication PCT No. WO 92/07065; Parault, et al., Nature, 1990, 344:565-569; Pieken, et al., Science, 1991, 253:314-317; Usman and Cedergren, Trends in Biochem. Sci., 1992, 17:334-339; Usman, et al., International Publication PCT No. WO 93/15197; Sproat, U.S. Pat. No. 5,334,711 and Beigelman, et al., 1995, J. Biol. Chem., 270:25702; all of the references are hereby incorporated in their totality by reference herein).

Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid catalysts of the instant invention.

Nucleic acid catalysts having chemical modifications which maintain or enhance enzymatic activity are provided. Such nucleic acid catalysts are also generally more resistant to nucleases than unmodified nucleic acid. Thus, in a cell and/or in vivo the activity may not be significantly lowered. As exemplified herein, such nucleic acid catalysts (e.g., VEGF-R-1 ribozymes) are useful in a cell and/or in vivo even if activity overall is reduced 10 fold (Burgin, et al., 1996, Biochemistry, 35:14090). Such ribozymes herein are said to “maintain” the enzymatic activity on all RNA ribozymes.

Therapeutic ribozymes delivered exogenously must optimally be stable within cells until translation of the target RNA has been inhibited long enough to reduce the levels of the undesirable protein. This period of time varies between hours to days depending upon the disease state. Clearly, ribozymes must be resistant to nucleases in order to function as effective intracellular therapeutic agents. Improvements in the chemical synthesis of RNA (Wincott, et al., 1995, Nucleic Acids Res., 23:2677; incorporated by reference herein) have expanded the ability to modify ribozymes by their nuclease stability as described above.

### V. Pharmaceutical Compositions: Ribozyme Delivery

In another embodiment, the present invention provides pharmaceutical compositions, the pharmaceutical compositions comprising a PEG-Cer formulated VEGF-R-1 ribozyme composition as described above and a pharmaceutically or veterinarially acceptable carrier. Such pharmacological compositions or formulations refer to a composition or formulation in a form suitable for administration, e.g., systemic administration or local administration, into a cell or patient, preferably a human. Suitable forms, in part, depend upon the use or the route of entry, for example oral, transdermal, or by injection. Such forms should not prevent the composition or formulation to reach a target cell (i.e., a cell to which the VEGF-R-1 ribozyme is being desired). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations, intravenous, subcutaneous, intraperitoneal, inhalation, oral, intrapulmonary and intramuscular. Each of these administration routes expose the desired ribozyme, to an accessible diseased tissue (Pavco, et al., 1997, IBC Conference on Strategies for Regulating Growth Factors, Jul. 14-15, 1997, Abstract). The rate of entry of a drug into the circulation has been shown to be a function of molecular weight or size. The use of a liposome or other drug carrier comprising the VEGF-R-1 ribozymes of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as the cancer cells.

As described above, the present invention provides compositions comprising a non-cationic lipid, a cationic lipid and a PEG-Cer conjugate. These formulations offer a method for increasing the accumulation of drugs, i.e., the VEGF-R-1 ribozymes, in target tissues. This class of drug carriers resists opsonization and elimination by the mononuclear phagocytic system (MPS or RES), thereby enabling longer blood circulation tunes and enhanced tissue exposure for the encapsulated drug. Such liposomes have been shown to accumulate selectively in tumors, presumably by extravasation and capture in the neovascularized target tissues. The long-circulating liposomes enhance the pharmacokinetics and pharmacodynamics of the VEGF-R-1 ribozymes, particularly compared to conventional cationic liposomes which are known to accumulate in tissues of the MPS (Liu, et al., J. Biol. Chem., 1995, 42:24864-24870; Choi, et al., International PCT Publication No. WO 96/10391; Ansell, et al., International PCT Publication No. WO 96/10390; Holland, et al., International PCT Publication No. WO 96/10392; all of these are incorporated by reference herein). Such long-circulating liposomes also protect the VEGF-R-1 ribozymes from nuclease degradation to a greater extent compared to cationic liposomes, based on their ability to avoid accumulation in metabolically aggressive MPS tissues such as the liver and spleen.y

The present invention also includes compositions suitable for administration or storage which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents may be provided. These include sodium benzoate, sorbic acid and esters of p-hydroxybenzoic acid. In addition, antioxidants and suspending agents may be used.

A pharmaceutically effective dose is that dose required to prevent, inhibit the occurrence, or treat (i.e., alleviate a symptom to some extent, preferably all of the symptoms) of a disease state. The pharmaceutically effective dose depends on the type of disease, the composition used, the route of administration, the type of mammal (e.g., patient) being treated, the physical characteristics of the specific mammal under consideration, concurrent medication, and other factors which those skilled in the medical arts will recognize. Generally, an amount between 0.01 mg/kg and 100 mg/kg body weight/day of active ingredients is administered dependent upon potency of the negatively charged polymer.

The term “patient” is used herein to refer to an organism which is a donor or recipient of explanted cells or the cells themselves. “Patient” also refers to an organism to which the compounds of the invention can be administered (e.g., locally through the use of a catheter or infusion pump, or systemically). Preferably, a patient is a mammal, e.g., a human, primate or a rodent.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### VI. EXAMPLES

### A. Example 1: Formation of Liposome Encapsulated Ribozyme Using the Reverse Phase Evaporation Method

Egg yolk phosphatiylcholine, cholesterol, and DOTAP was purchased from Avant) Polar Lipids (Albaster, Ala.). Equipment used in these examples were purchased from vendors, for example, an extruder was purchased from Lipex Biomembranes (Vancouver, B.C., Canada). An FPLC was purchased from Pharmacia (Piscataway, N.J.). A particle sizer was purchased from Malvern Instruments (Southborough, Mass.). PEG-Cer were synthesized as described in Choi, et al., 1996, supra, (incorporated by reference herein).

A mixture of a PEG-Cer, hammerhead ribozyme, phosphatidylcholine, cholesterol and a cationic lipid were formulated for animal studies. The following lipids suspended in chloroform were mixed together in a 50 mL round bottom flask: phosphatidylcholine (egg yolk) (190 mg), cholesterol (48.4 mg), DODAC (43.8 mg), PEG-Cer-C20 (133.8 mg) resulting in a molar ratio of 50:25:15:10. The lipids were dried down by rotary evaporation and then resuspended in ether (9 ml). A hammerhead ribozyme (mg) suspended in 1×phosphate buffered saline (3 ml) was added to the ether/lipid mixture and mixed together into an emulsion. In another preparation 1×PBS(3 ml) was used to form an empty vesicle control. Liposome vesicles were formed by removing the ether under vacuum. Residual ether was removed by bubbling argon gas through the lipid-ribozyme mixture for 10 minutes. Liposomes were then passed through a polycarbonate filter with 100 nm pores 6-10 times using an Extruder (Lipex Biomembranes, Vancouver, B.C.) with a 10 ml barrel. Vesicle diameter (120 nm) was confirmed using photon correlation spectroscopy (Malvern Instruments). Liposomes were purified from unencapsulated material using an FPLC column packed with DEAE sepharose CL-6B. Efficiency of encapsulation was determined by HPLC analysis an a C18 column (gradient of 4-18% acetonitrile in water). Lipid concentration was determined by measuring cholesterol concentration using a cholesterol quantitation assay (Sigma Chemicals) following the manufacturers instructions. In pharmacokinetic experiments the tritiated CHE (³H-cholesteryl hexadecyl ether) was used to track and quantitate lipid concentration and 32P was used to track the ribozyme concentration. Radioisotopes were quantitated in a scintillation counter.

### B. Example 2: Formation of Liposome Encapsulated Ribozyme by Bligh & Dyer Extraction

DOTAP (2.44 mg), EPC(2.75 mg), PEG-Ceramide-C8 (1.31 mg) were combined together suspended in chloroform in a glass test tube. The lipids were then dried down under argon gas. The lipid mixture was then suspended in a mixture of chloroform (0.73 ml) and Methanol (1.54 ml). A hammerhead ribozyme with a ³²P tracer (1 mg) suspended in water (0.73 ml) was then added to the lipid containing organic solvents. Vortexing the solution resulted in a monophasic solution of CHCl₃, MeOH and H₂O (1:2.1:1). Chloroform (0.75 ml) and water (0.75 ml) was then added to cause phase separation of the organic and aqueous components of the solution. The mix was then vortexed for 1 minute and then centrifuged at 2000 RPM for 5 minutes. The aqueous layer was then removed and then examined for ribozyme content by reading the absorbance at 260 nm wavelength using a spectrophotometer. The organic phase was dried down under argon gas and then rehydrated in normal saline. Ribozyme content was determined by counting a sample of the liposome preparation in a scintillation counter.

### C. Example 3: Pharmacokinetic Analysis of a Ribozyme-liposomal Formulation in Neonatal Murine Eyes

Seven day old (P7) neonatal mice and their nursing dams were placed into an oxygen rich chamber (75% O₂/25% N₂) with ad libitum food and water. Five days later (P12), they were removed from the chamber and injected immediately (day zero group) or allowed to recover five days and injected on P17 (day five group). Liposome formulated and non-formulated ribozyme was administered via intravitreal injection on P12 or P17. The neonatal mice, anesthetized with 40 μl 2.5% Avertin, received a single intravitreal bolus of 5 μg of VEGF-R-1 ribozyme (supplemented with 10×10⁵ cpm/μg ³²P VEGF-R-1 ribozyme; FIG. 2) formulated with EPC-DOTAP:PEG liposomes or non-formulated VEGF-R-1 ribozyme (supplemented with 10×10⁵ cpm/μg ³²P VEGF-R-1 ribozyme) in sterile saline. Neonates treated with ³²P VEGF-R-1 ribozyme were euthanized with CO₂ at 0.5, 4, 24, 48, 72 hours after ribozyme administration. Upon cessation of breathing, the chest cavity was opened and blood sampled (150-250 μl) from the heart. Sampled blood was added to a heparinized microfuge tube and centrifuged for 10 minutes to separate plasma and blood cells. Retina, capsule, kidney and liver were dissected from each and immediately frozen on dry ice. Frozen tissue from ³²P VEGF-R-1 ribozyme treated neonates was pulverized and digested in a proteinase K containing buffer (100 mM NaCl, 10 mM tris (pH 8), 25 mM EDTA, 10% SDS). A portion of the sample was added to scintillant and counted. Undiluted plasma was added to scintillant and counted. Tissue samples having greater than one hundred cpm per 50 μl of digested sample were analyzed for the presence and the percent of intact ribozyme via PAGE and phosphorimaging analysis.

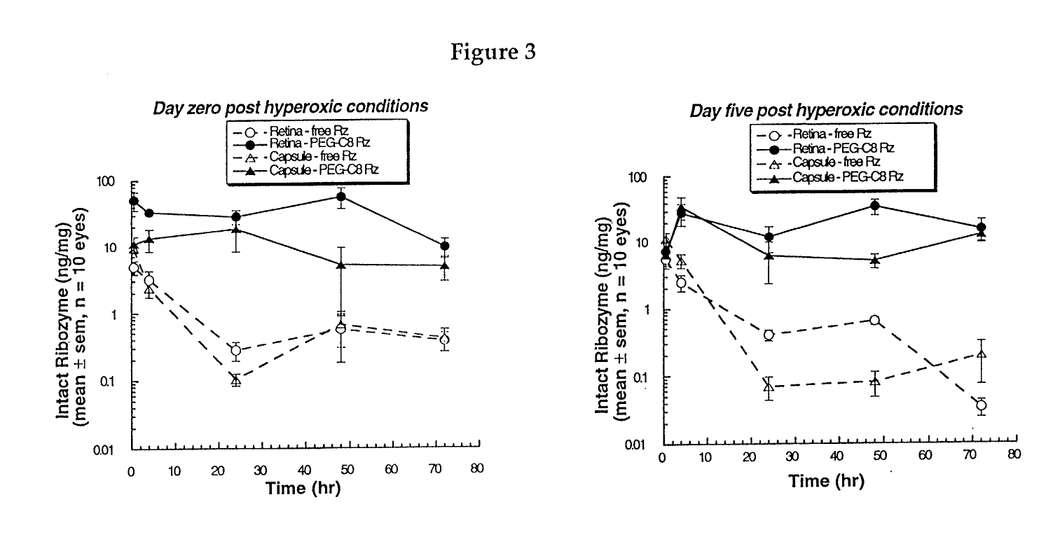

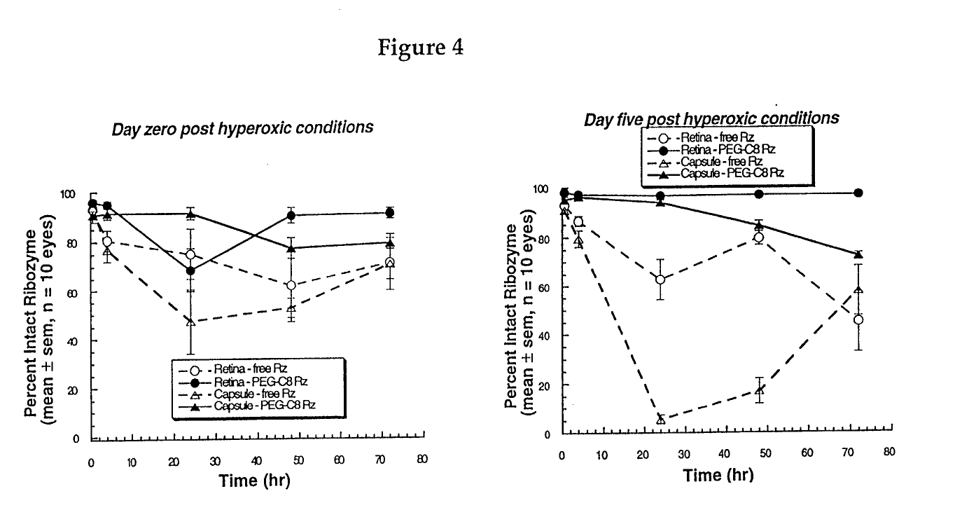

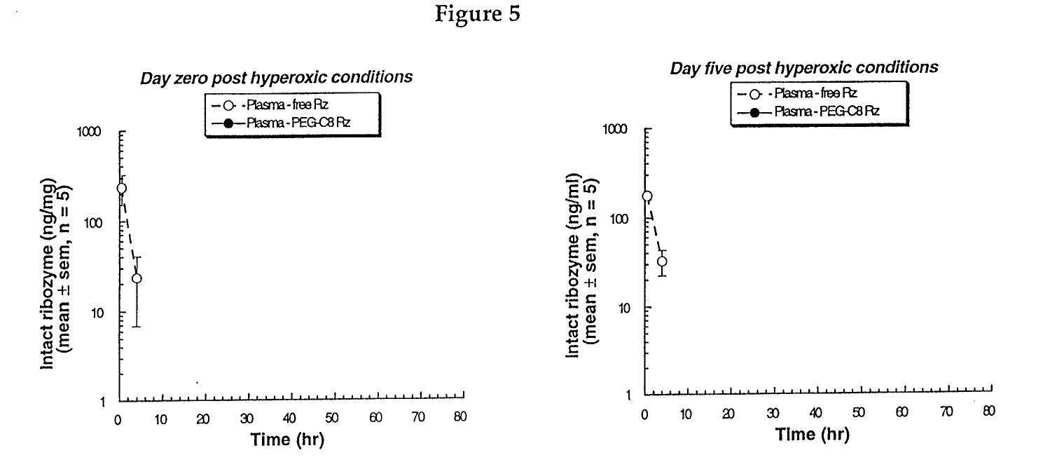

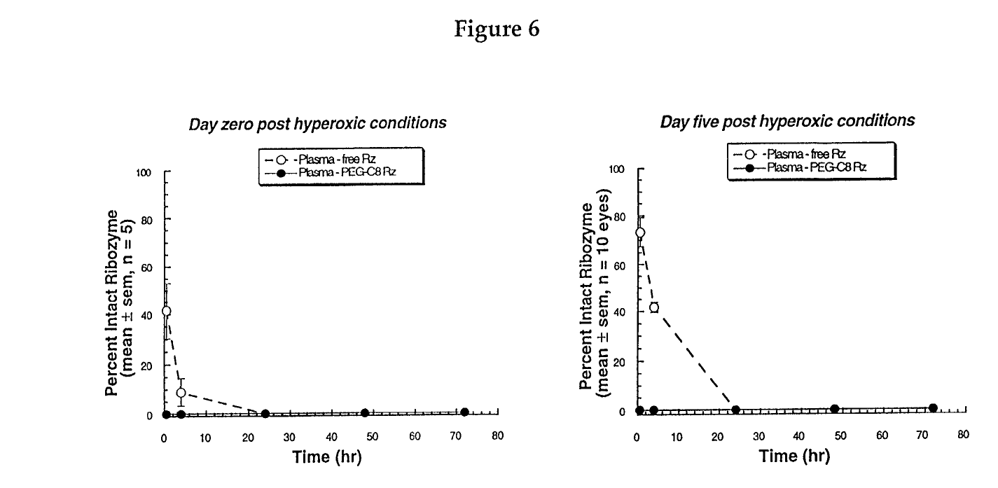

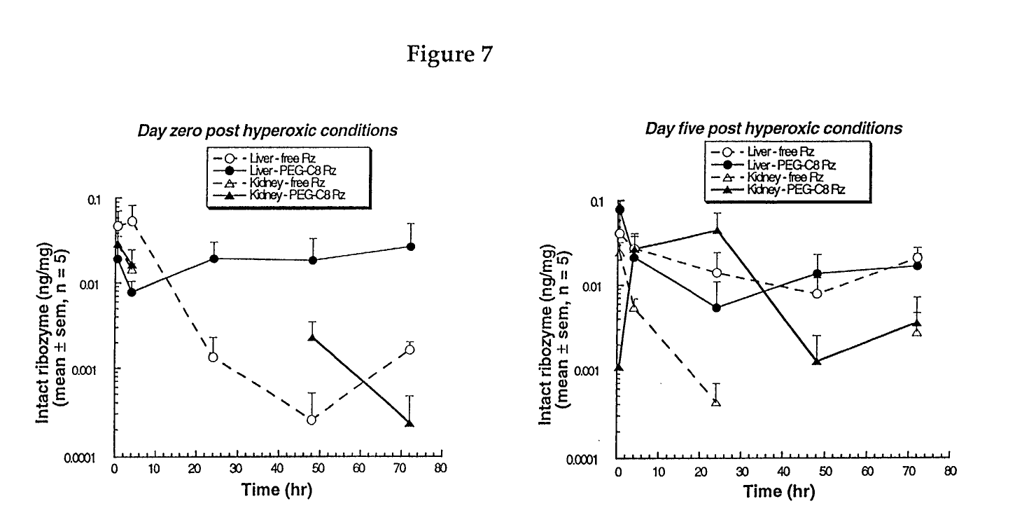

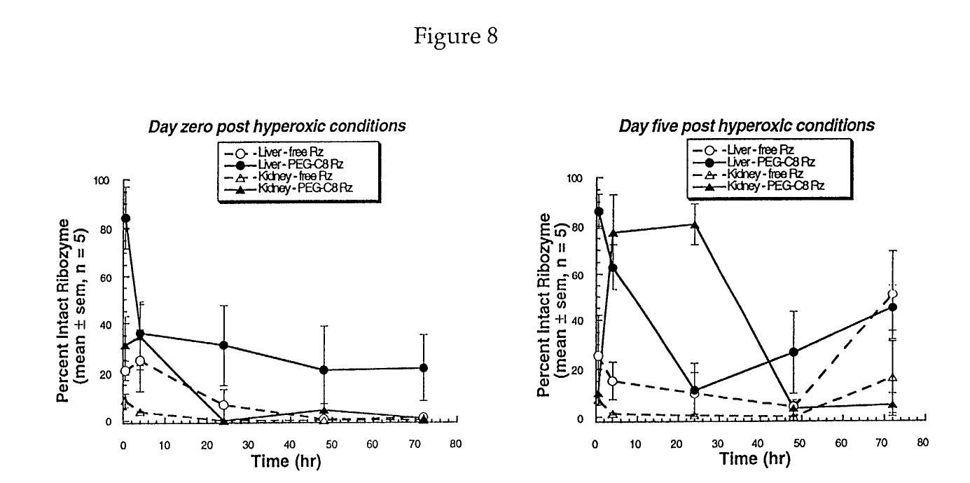

Concentrations of intact ribozyme in hyperoxic treated neonatal mouse retina and capsule are shown in FIG. 3. Intact ribozyme was detected in the retinas and capsules of the neonates through 72 hours (10 ng/mg) after injection of formulated ribozyme with 75-95% of the radioactivity associated with intact ribozyme (FIG. 4). Much lower concentrations of intact ribozyme were detected in the retina and capsule of the neonates administered free ribozyme (0.05-0.5 ng/mg at 72 hours. Concentrations of intact ribozyme in hyperoxic treated neonatal mouse plasma after intravitreal administration (on day zero and on day five) free or formulated ribozyme are shown in FIG. 5. Intact ribozyme was detected in plasma from animals treated with free ribozyme (15 ng/ml at 24 hours. However, there was no detectable intact ribozyme in the plasma of the neonates receiving liposome formulated ribozyme (FIG. 6). Tissue concentrations in the liver and kidney after intravitreal injection of formulated or free ribozyme are shown in FIG. 7. Intact ribozyme was detected in the livers of the neonates 72 hours after injection of formulated ribozyme (0.05 ng/mg) or free ribozyme (0.001 ng/mg). In kidneys of the neonates in the day zero group, intact ribozyme was detected only through the 4 hour time point (0.03 ng/mg) after administration of free ribozyme. However, intact ribozyme was detected in kidneys through 4 hours and then again at the 48 and 72 hours after administration of formulated ribozyme (FIG. 8).

Area under the concentration time curve (AUC) was calculated as an indication of tissue ribozyme exposure. As shown in Table II, there was a 25 to 37 fold increase in the AUC over the 72 hour time course when the injected ribozyme was formulated with EYPC:DOTAP-PEG C8 liposomes compared with free ribozyme. There was also a 9 to 11 fold increase in ribozyme exposure of the capsule with the formulated ribozyme. AUC calculations for kidney, liver and plasma were not performed due to intermittent detection of intact ribozyme.

TABLE II
Retina and capsule areas under the curve (AUC) from hyperoxic treated
meonatal mouse ribozyme tissue concentrations after intravitreal administration of
5 μg VEGF-R-1 ribozyme (supplemented with 10 × 10⁶ cpm ³²P VEGF-R-1)
formulated in an EYPC:DOTAP:PEG liposome or non-formulated (EYPC =
egg yolk phosphatidylcholine). Mice were administered ribozyme either immediately
upon their removal from the hyperoxic chamber or five days after their removal
from the hyperoxic chamber.

| | | Day 0 | | Day 5 | |
|---|---|---|---|---|---|
| | | | PEG-C8 AUC | | PEG-C8 AUC |
| Tissue | Formulation | AUC_{0-72 hr} | Free AUC | AUC_{0-72 hr} | Free AUC |
| Retina | Free | 71 | 37 | 65 | 25 |
| | PEG-C8 | 2600 | | 1649 | |
| Capsule | Free | 70 | 11 | 91 | 9 |
| | PEG-C8 | 740 | | 850 | |
| Plasma | Free | 515 | | 413 | |
| | PEG-C8 | ND | | ND | |

### D. Example 4. Blood Clearance Screen of Intravenously Administered Liposomal Formulations

Female C57B1/6J weighing 20-25 g were used to screen various formulations of liposome encapsulated ribozyme. The following formulations were prepared using the protocol in example 1: EPC:CHOL (55:45), Shingomyelin(SM):EPC:CHOL (33:33:33), and EPC:CHOL:DODAC:PEG-ceramide-C20 (50:25:15:10). In these experiments the ribozyme included a tracer of ³²P labeled ribozyme and CHE was used to track and quantitate the lipid. A single i.v. made via the tail vein. Each dose contained about 3 μmoles total lipid and between 25 50 μg of VEGF-R-1 ribozyme in a volume of 100 μL. The time points observed were 15 minutes, 2 hours, 4 hours and 24 hours. At each time point animals were euthanized with CO₂. Upon cessation of breathing, the chest cavity was opened and blood sampled (200-500 μL) from the heart. Sampled blood was added to a heparinized microfuge tube and centrifuged for 10 min to separate plasma and blood cells. Plasma samples were treated with proteinase K containing buffer. A portion of the sample was added to scintillant and counted. The sample was resolved via 15% polyacrylamide gel electrophoresis and quantitated using phosphorimager analysis.

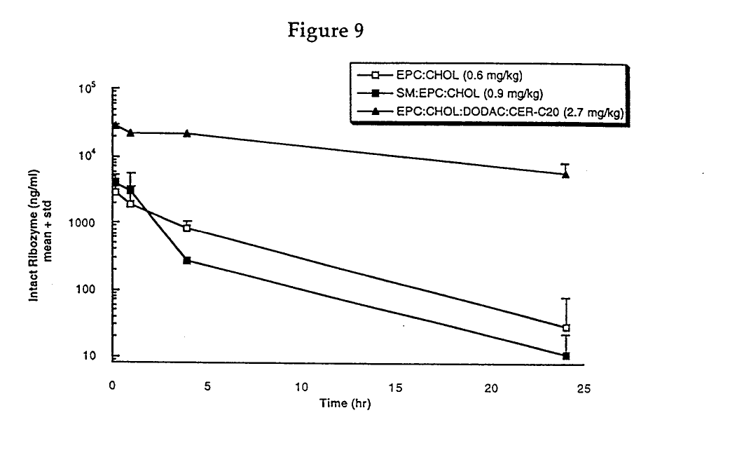

The data (FIG. 9) indicated that of the three formulations tested, the best was the formulation which contained PEG-Ceramide. The PEGylated liposomes were present in large quantities even after 24 hours suggesting that the elimination half life may be in the order of hours if not days.

### E. Example 5: Pharmacokinetic Evaluation of Liposome Encapsulated Ribozymes in Lewis Lung Carcinoma Model

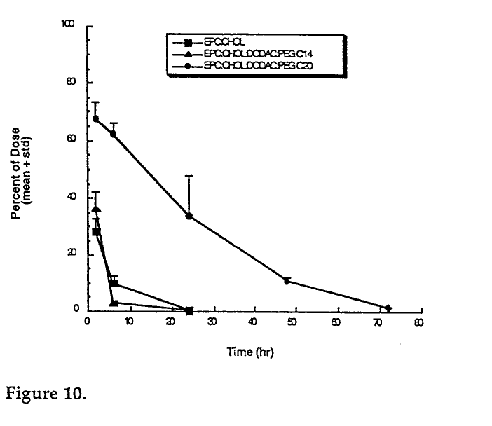

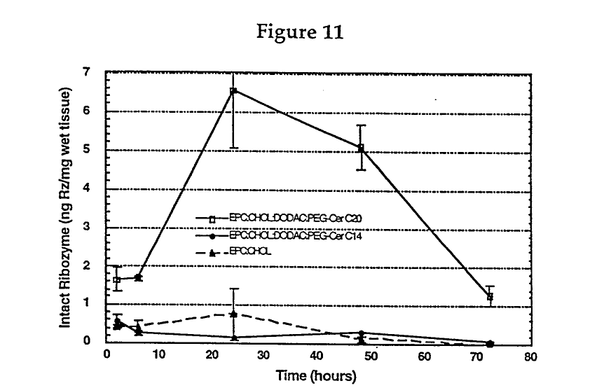

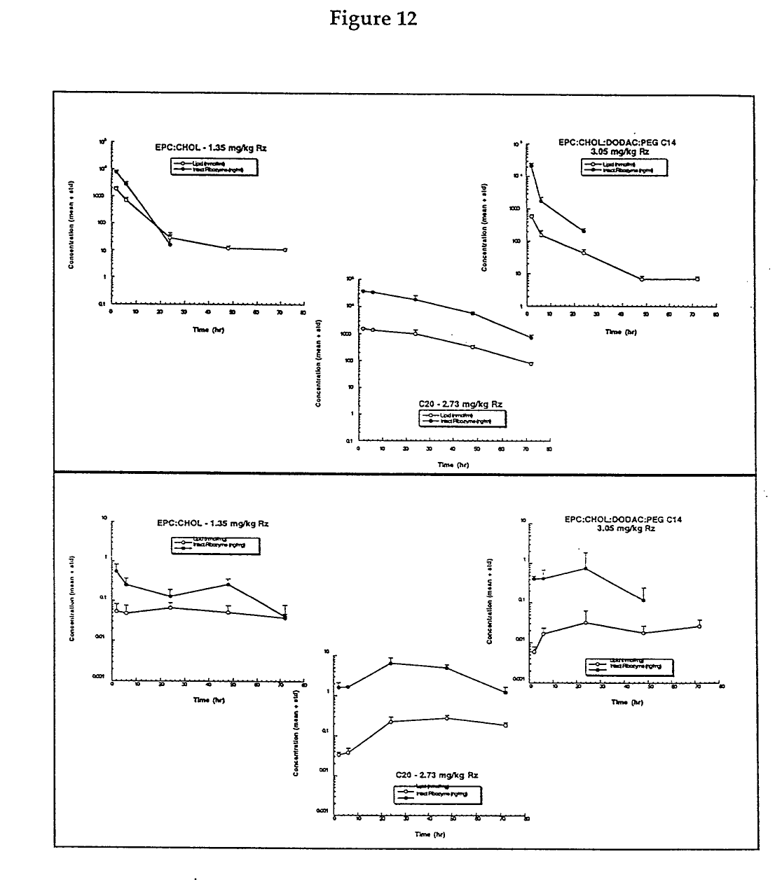

Female C57B1/6J weighing 20-25 g were implanted with a 0.1 mL suspension of Lewis Lung carcinoma tumor cells (5×10⁶ cells/mL in normal saline), injected subcutaneously into the right flank. Tumors were allowed to grow for 17 days prior to dosing with liposomal ribozyme formulations. Formulations were made using the protocol described in example 1. EPC:CHOL:DODAC:PEG-ceramide-C20 (50:25:15:10), EPC:CHOL:DODAC:PEG-ceramide-C8 (50:25:15:10) and EPC:CHOL liposomes were made with CHE as a tracer. Ribozyme contained ³²P labeled ribozyme tracer. A single i.v. bolus injection was made via the tail vein. Injections may also be made via the jugular vein. Each “liposome formulation” dose contained about 3 μmoles total lipid and between 25-50 μg of VEGF-R-1 ribozyme in a volume of 100 μL. After dosing and at the indicated harvest times (2, 6, 24, 48, and 72 hours), animals were euthanized with CO₂. Upon cessation of breathing, the chest cavity was opened and blood sampled (200-500 μL) from the heart. Sampled blood will be added to a heparinized microfuge tube and centrifuged for 10 minutes to separate plasma and blood cells. Following blood sampling, animals were perfused with sterile saline through the heart until the liver is cleared of blood (10 mL). The tumor and the adjacent vascular tissue were surgically removed, snap frozen in liquid nitrogen and transferred to a tared culture tube. Tissue was then pulverized or homogenized and then digested with proteinase K containing buffer. A portion of the sample was added to scintillant and counted. The sample was analyzed via PAGE and phosphorimaging. Liposomes containing PEG-Cer-C20 lipid performed better than PEG-Cer-C14 or EPC:CHOL liposomes, based on plasma levels of intact ribozyme (FIG. 10). On the other hand, the data for the PEG-Cer-C20 containing liposome about 7% of the administered ribozyme dose was detected as intact ribozyme in plasma after 72 h. Tumor exposure was significantly enhanced for PEG-ceramide-C20 containing liposomal formulations compared to the other ribozyme formulations (FIG. 11). The degree of enhancement correlated roughly with plasma levels (FIG. 9). Quantitations of ³²P-ribozyme and ³H-CHE lipid tracer indicated that the liposomes circulate in blood mostly intact with minimal degradation. Similar clearance profiles were also observed in primary tumor tissue (FIG. 12).

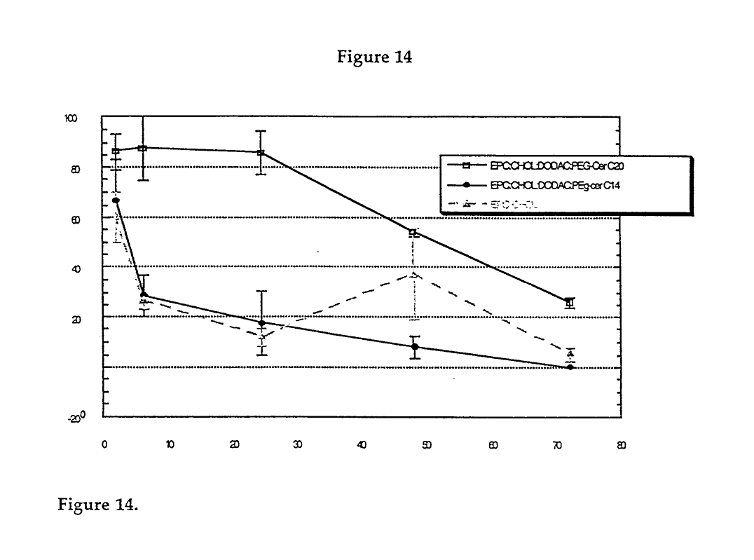

Ribozyme stability in tumor tissue was measured after resolving samples by polyacrylamide gel electrophoresis (PAGE) as described above. Stability was measured as the percent of total radioactivity that still remained as full length ribozyme. Ribozymes delivered using PEG-cer-C20 liposomes were 85-90% intact through 24 hours. The ribozymes delivered using the other two formulations were approximately 30% intact after just 6 hours (FIG. 14).

### F. Example 6: Ribozyme-efficacy in C57 Mice

Sustained tumor growth and metastasis depend upon angiogenesis. In fact, the appearance of vessels in a growing tumor is correlated with the beginning of metastatic potential. Several studies have shown that antiangiogenic agents alone or in combination with cytotoxic agents reduce lung metastases and/or primary tumor volume in the Lewis lung and B-16 melanoma models (Bergstrom, et al., 1995, Anticancer Res., 15:719-728; Kato, et al., 1994, Cancer Res., 54:5143-5147; O'Reilly, et al., 1994, Cell, 79:315-328; Sato, et al., 1995, Jpn. J. Cancer Res., 86:374-382).

A major factor implicated in the induction of solid tumor angiogenesis is vascular endothelial growth factor (VEGF; Folkman, 1995, supra). Several human tumors have been shown to synthesize and secrete. With regard to treating lung metastasis, VEGF and VEGF receptors of both subtypes and their expression are upregulated in the lung under conditions of hypoxia (Tuder, et al., 1994, J. Clin. Invest., 95:1798-1807). This may lead to neovascularization which provides the means by which tumor cells gain access to circulation (Mariny-Baron and Marmé, 1995). Thus, VEGF and its receptors may be important targets in the treatment of metastatic disease.

It has recently been shown that a catalytically active ribozyme targeting flt-1 RNA inhibits VEGF-induced neovascularization in a dose-dependent manner in a rat cornmeal model of angiogenesis (Cushman, et al., 1996, Angiogenesis Inhibitors and Other Novel Therapeutic Strategies for Ocular Diseases of Neovascularization, IBC Conference Abstract). Testing with cytotoxic agents in combination with antiangiogenic ribozymes (for example VEGF-R-1 ribozyme; FIG. 1) may also prove useful.

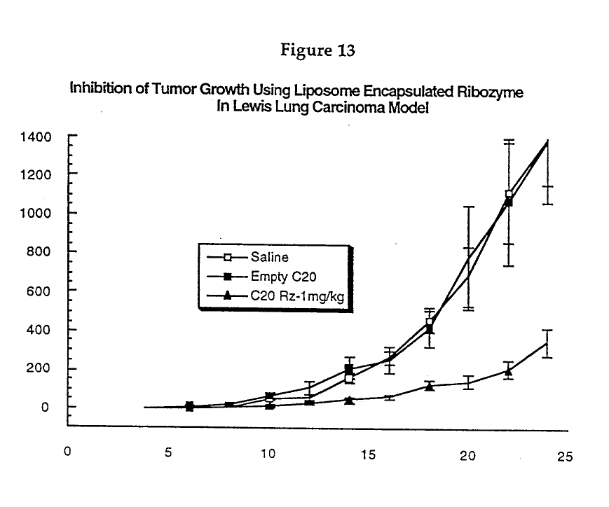

C57/BL6 female mice were instrumented with jugular catheters three days, after receiving a subcutaneous inoculation of 5×10⁵ cells Lewis lung carcinoma cells (highly metastatic variant) in a volume of 0.1 ml saline. Catheters (PE50) were implanted in the jugular vein and exteriorized for daily bolus administration. Each dose of EPC:Cholesterol:PEG-Cer-C20:DODAC (50:25:15: 10) formulated VEGF-R-1 ribozyme offered to the mice was 1 mg ribozyme/kg body wt. The liposome formulation was prepared using the Reverse Phase Evaporation method. Liposomes were injected by a hamilton syringe into the catheter and the catheter tubing was flushed using 100 μl of saline. Animals were not treated on days 18-25 after tumor implantation. Tumors were measured with a microcaliper on days 2-25 every other day to determine tumor growth. Tumor volume was determined by the following formula: [length*(width)²]/2. Twenty five days following inoculation, animals were euthanized and tumors removed and weighed. To preserve tumors for possible quantitation of ribozyme content, tumors were quickly frozen in liquid nitrogen and stored at −70° C. Lungs were removed and weighed and macrometastasis counted under 4×magnification using a Leitz dissecting microscope. The data as shown in FIG. 13 indicates that liposome encapsulated ribozyme inhibited tumor growth during the duration of dosing. Following cessation of ribozyme dosing the data suggests an increase in the rate of tumor growth.

### G. Example 7: Exemplar Ribozymes

This example illustrates the characteristics of naturally occurring ribozymes.

Group I Introns

Size: ˜150 to ˜1000 nucleotides.

Requires a U in the target sequence immediately 5′ of the cleavage site.

Binds 4-6 nucleotides at the 5′-side of the cleavage site.

Reaction mechanism: attack by the 3′-OH of guanosine to generate cleavage products with 3′-OH and 5′-guanosine.

Additional protein cofactors required in some cases to help folding and maintenance of the active structure.

Over 300 known members of this class. Found as an intervening sequence in Tetrahymena thermophila rRNA, fungal mitochondria, chloroplasts, phage T4, blue-green algae, and others.

Major structural features largely established through phylogenetic comparisons, mutagenesis, and biochemical studies [¹].

Complete kinetic framework established for one ribozyme [^{2,3,4,5}].

Studies of ribozyme folding and substrate docking underway [^{6,7,8}].

Chemical modification investigation of important residues well established [^{9,10}].

The small (4-6 nt) binding site may make this ribozyme too non-specific for targeted RNA cleavage, the Tetrahymena group I intron has been used to repair a “defective” β-galactosidase message by the ligation of new β-galactosidase sequences onto the defective message [¹¹].

RNAse P RNA (M1 RNA)

Size: ˜290 to 400 nucleotides.

RNA portion of a ubiquitous ribonucleoprotein enzyme.

Cleaves tRNA precursors to form mature tRNA [¹²].

Reaction mechanism: possible attack by M²⁺—OH to generate cleavage products with 3′-OH and 5′-phosphate.

RNAse P is found throughout the prokaryotes and eukaryotes. The RNA subunit has been sequences from bacteria, yeast, rodents and primates.

Recruitment of endogenous RNAse P for therapeutic applications is possible through hybridization of an External Guide Sequence (EGS) to the target RNA [^{13,14}].

Important phosphate and 2′ OH contacts recently identified [^{15,16}].

Group II Introns

Size: ˜1000 nucleotides.

Trans cleavage of target RNAs recently demonstrated [^{17,18}].

Sequence requirements not fully determined.

Reaction mechanism: 2′-OH of an internal adenosine generates cleavage products with 3′-OH and a “lariat” RNA containing a 3′-5′ and a 2′-5′ branch point.

Only a natural ribozyme with demonstrated participation in DNA cleavage [^{19,20}] in addition to RNA cleavage and ligation.

Major structural features largely established through phylogenetic comparisons [²¹].

Important 2′ OH contacts beginning to be identified [²²].

Kinetic framework under development [²³].

Neurospora VA RNA

Size: ˜144 nucleotides.

Trans cleavage of hairpin target RNAs recently demonstrated [²⁴].

Sequence requirements not fully determined.

Reaction mechanism: attack by 2′-OH5′ to the scissile bond to generate cleavage products with 2′,3′-cyclic phosphate and 5′-OH ends.

Binding sites and structural requirements not fully determined.

Only 1 known member of this class. Found in Neorospora VS RNA.

Hammerhead Ribozyme

Size: ˜13 to 40 nucleotides.

Requires the target sequence UH immediately 5′ of the cleavage site.

Binds a variable number nucleotides on both sides of the cleavage site.

Reaction mechanism: attack by 2′-OH5′ to the scissile bond to generate cleavage products with 2′,3′-cyclic phosphate and 5′-OH ends.

14 known members of this class. Found in a number of plant pathogens (virusoids) that use RNA as the infectious agent.

Essential structural features largely defined, including 2 cystal structures [^{25,26}].

Minimal ligation activity demonstrated (for engineering through in vitro selection) [²⁷].

Complete kinetic framework established for two or more ribozymes [²⁸].

Chemical modification investigation of important residues well established [²⁹].

Hairpin Ribozyme

Size: ˜50 nucleotides.

Requires the target sequence GUC immediately 3′ of the cleavage site.

Binds 4-6 nucleotides at the 5′-side of the cleavage site and a variable number to the 3′-side of the cleavage site.

Reaction mechanism: attack by 2′-OH5′ to the scissile bond to generate cleavage products with 2′,3′-cyclic phosphate and 5′-OH ends.

3 known members of this class. Found in three plant pathogen (satellite RNAs of the tobacco ringspot virus, arabis mosaic virus and chicory yellow mottle virus) which uses RNA as the infectious agent.

Essential structural features largely defined [^{30,31,32,33}].

Ligation activity (in addition to cleavage activity) makes ribozyme amendable to engineering through in vitro selection [³⁴].

Complete kinetic framework established for one ribozyme [³⁵].

Chemical modification investigation of important residues begun [36,37].

Hepatitis Delta Virus (HDV) Ribozyme

Size: ˜60 nucleotides.

Trans cleavage of target RNAs demonstrated [³⁸].

Binding sites and structural requirements not fully determined, although no sequences 5′ of cleavage site are required. Folded ribozyme contains a pseudoknot structure [³⁹].

Reaction mechanism: attack by 2′-OH5′ to the scissile bond to generate cleavage products with 2′,3′-cyclic phosphate and 5′-OH ends.

Only 2 known members of this class. Found in human HDV.

Circular form of HDV is active and shows increased nuclease stability [⁴⁰].

1. Michel, F., et al., Nat. Sruct. Biol., Vol 1(1).5-7 (1994). Lisacek, F., et al., J. Mol. Biol., Vol. 235(4):1206-17 (1994).

2. Herschlag, D., et al., Biochemistry, Vol. 29(44):10159-71 (1990).

3. Herschlag, D., et al., Biochemistry, Vol. 29(44):10172-80 (1990).

4. Knitt, D., et al., Biochemistry, Vol. 35(5):1560-70 (1996).

5. Bevilacqua, P., et al., Biochemistry, Vol. 35(2):648-58 (1996).

6. Li, Y., et al., Biochemistry, Vol. 34(44):14394-9 (1995).

7. Banerjee, A., et al., Biochemistry, Vol. 34(19):6504-12 (1995).

8. Zarrinkar, P., et al., Nucleic Acids Res., Vol. 24(5):854-8 (1996).

9. Strobel, S., et al., Science, Vol. 267(5198):675-9 (1995).

10. Strobel, S., et al., Biochemistry, Vol. 35(4):1201-11 (1996).

11. Sullenger, B., et al., Nature, Vol. 371(6498):619-22 (1994).

12. Robertson, H., et al., J. Biol. Chem., Vol. 247:5243-5251 (1972).

13. Forster, A., et al., Science, Vol. 249(4970):783-6 (1990).

14. Yuan, Y., et al., Proc. Natl. Acad. Sci. USA, Vol. 89:8006-10 (1992).

15. Harris, M., et al., RNA, Vol. 1(2):210-18 (1995).

16. Pan, T., et al., Proc. Natl. Acad. Sci. USA, Vol. 92(26):12510-14 (1995).

17. Pyle, A., et al., Biochemistry, Vol. 33(9):2716-25 (1994).

18. Michels, W., et al., Biochemistry, Vol. 34(9):2965-77 (1995).

19. Zimmerly, S., et al., Cell, Vol. 83(4):529-38 (1995).

20. Griffin, E., et al., Chem. Biol., Vol. 2(11):761-70 (1995).

21. Michel, F., et al., Annu. Rev. Biochem., Vol. 64:435-61 (1995).

22. Abramovitz, D., et al., Science, Vol. 271(5254):1410-13 (1996).

23. Daniels, D., et al., J. Mol. Biol., Vol. 256(1):31-49 (1996).

24. Guo, H., et al., EMBO J., Vol. 14(2):368-76 (1995).

25. Scott, W., et al., Cell, Vol. 81:991-1002 (1995).

26. McKay, RNA, Vol. 2:395-403 (1996).

27. Long, D., et al., U.S. Pat. No. 5,633,133.

28. Hertel, K., et al., Biochem., Vol. 33:3374-3385 (1994). Beigelman, L., et al., J. Biol. Chem., Vol. 270:25702-25708 (1995).

29. Beigelman, L., et al., J. Biol. Chem., Vol. 270:25702-25708 (1995).

30. Hampel, A., et al., Nucleic Acids Res., Vol. 18(2):299-304 (1990).

31. Chowrira, B., et al., Nature, Vol. 354(6351):320-2 (1991).

32. Berzal-Herranz, A., et al., EMBO J., Vol. 12(6):2567-73 (1993).

33. Joseph, S., et al., Genes Dev., Vol. 7(1):130-8 (1993).

34. Berzal-Herranz, A., et al., Genes Dev., Vol. 6(1):129-34 (1992).

35. Hegg, L., et al., Biochemistry, Vol. 34(48):15813-28 (1995).

36. Grasby, J., et al., Biochemistry, Vol. 34(12):4068-76 (1995).

37. Schmidt, S., et al., Nucleic Acids Res., Vol. 24(4):573-81 (1996).

38. Perrotta, A., et al., Biochemistry, Vol. 31(1):16-21 (1992).

39. Perrotta, A., et al., Nature, Vol. 350(6317):434-6 (1991).

40. Puttaraju, M., et al., Nucleic Acids Res., Vol. 21(18):4253-8 (1993).

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated herein by reference for all purpose.

What is claimed is:

1. A composition for facilitating delivery of a nucleic acid catalyst to a biological system, said composition comprising a polyethylene glycol (PEG)-ceramide conjugate, a lipid and said nucleic acid catalyst in proportions sufficient to achieve said delivery of said nucleic acid catalyst to said biological system.

2. The composition of claim 1 further comprising phosphatidyl choline.

3. The composition of claim 1 further comprising cholesterol.

4. The composition of claim 1 further comprising phosphatidyl choline and cholesterol.

5. The composition of claims 1, 2, 3 or 4, wherein said nucleic acid catalyst has an endonuclease activity.

6. The composition of claim 5, wherein said nucleic acid catalyst comprises one or more ribonucleotides.

7. The composition of claim 5, wherein said nucleic acid catalyst comprises one or more deoxyribonucleotides.

8. The composition of claim 5, wherein said nucleic acid catalyst is in a hammerhead motif.

9. The composition of claims 1, 2, 3 or 4, wherein said lipid is a cationic lipid.

10. The composition of claims 1, 2, 3 or 4, wherein said lipid is N,N-dioleyl-N,N-dimethylammonium chloride (DODAC).

11. The composition of claims 1, 2, 3 or 4, wherein said lipid is 1,2-dioleoyloxy-3(N,N,N-trimethylamino)propane chloride (DOTAP).

12. The composition of claims 1, 2, 3 or 4, wherein said PEG-Ceramide conjugate comprises a fatty acid group having eight carbon atoms.

13. The composition of claims 1, 2, 3 or 4, wherein said PEG-Ceramide conjugate comprises a fatty acid group having fourteen carbon atoms.

14. The composition of claims 1, 2, 3 or 4, wherein said PEG-Ceramide conjugate comprises a fatty acid group having twenty carbon atoms.

15. The composition of claims 2 or 4, wherein said phosphatidyl choline is egg yolk phosphatidyl chorine.

16. A pharmaceutical composition comprising the composition of claims 1, 2, 3 or 4 and a pharmaceutically or veterinarially acceptable carrier.

17. A mammalian cell comprising the composition of claims 1, 2, 3 or 4.

18. The mammalian cell of claim 17, wherein said mammalian cell is a human cell.

19. A mammalian cell comprising the pharmaceutical composition of claim 16.

20. The mammalian cell of claim 19, wherein said mammalian cell is a human cell.

21. The composition of claims 1, 2, 3 or 4, wherein said nucleic acid catalyst is capable of decreasing the expression of RNA associated with a mammalian disease.

22. The composition of claim 21, wherein said mammalian disease is a human disease.

23. The composition of claim 21, wherein said disease is cancer.

24. The composition of claim 21, wherein said disease is inflammation.

25. A pharmaceutical composition comprising the composition of claim 21 and a pharmaceutically or veterinarially acceptable carrier.

26. A method of facilitating the transfer of a nucleic acid catalyst into a cell, said method comprising contacting said cell with the composition of claims 1, 2, 3 or 4 under conditions suitable for the transfer of said nucleic acid catalyst into said biological system.

27. A method of treatment of a disease in a patient, said method comprising administering to said patient the pharmaceutical composition of claim 25 under conditions in which the expression the RNA associated with said disease is decreased in said patient and a therapeutic result is attained.

28. The method of claim 27, wherein said disease is cancer.

29. The method of claim 27, wherein said disease is inflammation.

30. The method of claim 27, wherein said administration is a systemic administration.

31. A method of treatment of a disease in a patient comprising the step of administering to said patient the composition of claim 21 under conditions in which the expression the RNA associated with said disease is decreased in said patient and a therapeutic result is attained.

32. The method of claim 31, wherein said disease is cancer.

33. The method of claim 31, wherein said disease is inflammation.

34. The method of claim 31, wherein said administration is a systemic administration.

35. The composition of claims 1, 2, 3 or 4, wherein said nucleic acid catalyst is chemically modified.

36. The composition of claim 5, wherein said nucleic acid catalyst specifically cleaves RNA encoded by vascular endothelial growth factor receptor (VEGF-R) RNA.

37. The composition of claim 36, wherein said nucleic acid catalyst is VEGF-R-1.

38. The pharmaceutical composition of claim 16 further comprising pharmaceutically acceptable fillers, adjuvants and diluents.

39. A method of cleaving a merger nucleic acid molecule in a cell, said method comprising contacting said cell with the composition of claim 5 under conditions suitable for the cleavage of said merger nucleic acid molecule.

40. The composition of claims 1, 2, 3 or 4, wherein said composition is formed by the reverse phase evaporation process.

41. The composition of claims 1, 2, 3 or 4, wherein said composition is formed by the Bligh and Dyer extraction method.

42. The composition of claims 1, 2, 3 or 4, wherein the concentration of said lipid is between 0-30 percent.

43. The composition according to claim 42, wherein the concentration of said lipid is between 5-30 percent.

44. The composition of claim 43, wherein the concentration of said lipid is 15 percent.

45. The composition of claim 15, wherein the concentration of said egg yolk phosphatidyl choline is 50 percent, the concentration of said cholesterol is 25 percent, the concentration of said lipid is 15 percent and the concentration of said PEG-Ceramide conjugate is 10 percent.

46. The composition of claims 1, 2, 3 or 4, wherein said nucleic acid catalyst is represented by a plasmid expression vector encoding said nucleic acid catalyst ia a manner that allows expression of said nucleic acid catalyst in said biological system.

47. The composition of claims 1, 2, 3 or 4, wherein said biological system is a tumor.

48. The composition of claims 1, 2, 3 or 4, wherein said biological system is a mammalian eye.

49. The composition of claims 1, 2, 3 or 4, wherein said PEG-Ceramide conjugate comprises a fatty acid group having between six and twenty carbon atoms.

50. A composition for facilitating delivery of a nucleic acid catalyst to a biological system, said method comprising a polyethylene glycol (PEG)-ceramide conjugate, phosphatidylcholine, cholesterol and said nucleic acid catalyst in proportions sufficient to achieve said delivery of the nucleic acid catalyst to said biological system.

51. The composition of claim 50, wherein said nucleic acid catalyst has an endonuclease activity.

52. The composition of claim 50, wherein said nucleic acid catalyst comprises one or more ribonucleotides.

53. The composition of claim 50, wherein said nucleic acid catalyst comprises one or more deoxyribonucleotides.

54. The composition of claim 50, wherein said nucleic acid catalyst is in a hammerhead motif.

55. The composition of claim 50, wherein said PEG-Ceramide conjugate comprises a fatty acid group having between six and twenty carbon atoms.

56. The composition of claim 55, wherein said PEG-Ceramide conjugate comprises a fatty acid group having eight carbon atoms.

57. The composition of claim 55, wherein said PEG-Ceramide conjugate comprises a fatty acid group having fourteen carbon atoms.

58. The composition of claim 55, wherein said PEG-Ceramide conjugate comprises a fatty acid group having twenty carbon atoms.

59. The composition of claim 50, wherein said phosphatidyl choline is egg yolk phosphatidyl choline.

60. A pharmaceutical composition comprising the composition of claim 50 and a pharmaceutically or veterinarially acceptable carrier.

61. A composition for facilitating the delivery of a nucleic acid catalyst to a biological system, said composition comprising a non-cationic lipid, a cationic lipid, a polyethyleneglycol-ceramide (PEG-Cer) conjugate and said nucleic acid catalyst in proportions sufficient to achieve the delivery of said nucleic acid catalyst to said biological system.
